(19) United States
(12) Patent Application Publication
(10) Pub. No.: US 2026/0015561 A1
(43) Pub. Date: Jan. 15, 2026

(54) DRUM-SHAPED CONTAINER RACK FOR BLOOD CULTURE BOTTLES AND METHODS OF MEASUREMENT AND OPERATION

(71) Applicant: BECTON, DICKINSON AND COMPANY, Franklin Lakes, NJ (US)

(72) Inventors: Brent Ronald Pohl, Timonium, MD (US); Kevin Wenger, Baltimore, MD (US); Stephen Robert LaChance, Cockeysville, MD (US); Daniel Justin Lohan, Glen Rock, PA (US); Dwight Livingston, Fallston, MD (US); Robert Edward Armstrong, Hunt Valley, MD (US); Edward M. Skevington, Stewartstown, PA (US); Jingyun Zhang, Freeland, MD (US); Johnny Alberto Quinteros, Mc Sherrystown, PA (US)

(73) Assignee: BECTON, DICKINSON AND COMPANY, Franklin Lakes, NJ (US)

(21) Appl. No.: 18/994,741

(22) PCT Filed: Jul. 18, 2023
(86) PCT No.: PCT/US2023/027989

§ 371 Date: Jan. 15, 2025

Related U.S. Application Data
(60) Provisional application No. 63/390,452, filed on Jul. 19, 2022.
(60) Provisional application No. 63/432,460, filed on Dec. 14, 2022.

(51) Int. Cl. C12M 1/24; C12M 1/00; C12M 3/00; C12M 3/04

(52) U.S. Cl. CPC C12M 23/08; C12M 23/48; C12M 27/10; C12M 41/14

(57) ABSTRACT

An apparatus for storing and monitoring blood culture bottles. The apparatus has a moveable rack configured as a drum having a plurality of receptacles therein for receiving blood culture bottles. The drum is disposed in a housing. The housing includes a heater and a blower for incubating the blood culture bottles at elevated temperatures. The receptacles have a light pipe to provide an indication of bottle status as determined by the apparatus. The apparatus has a housing with a plurality of cabinets therein. Each cabinet has a molded foam insert heat distribution assembly therein. Each molded foam insert heat distribution assembly is in fluid communication with a heater/blower assembly. The apparatus also may have a drum motor encoder affixed to a motor placed in the center of the moveable rack, which is drum-shaped. The motor encoder detects if the drum-shaped rack is in or out of alignment and increments manual or automatic rotation of the drum-shaped rack.

### CROSS REFERENCE TO RELATED APPLICATIONS

This application claims priority to and the benefit of U.S. Provisional Application No. 63/432,460 which was filed on Dec. 14, 2022. This application also claims the benefit of and is related to U.S. Provisional Application No. 63/390,452, filed Jul. 19, 2022. Both provisional applications are incorporated by reference herein. This application is also related to PCT Application No. PCT/US2020/045065, which was filed Aug. 5, 2020, and which is incorporated by reference herein.

### TECHNICAL FIELD

The present invention relates to a non-invasive apparatus for detecting biological activities in a specimen such as blood, where a number of specimens with culture medium are introduced into a large number of sealable containers and are exposed to conditions enabling a variety of metabolic, physical, and chemical changes to take place in the presence of microorganisms in the sample. These changes are then monitored using calorimetric or fluorescent chemical sensors disposed to the inner bottom of each blood culture bottle as the bottles are rotated in a rotatable drum. After monitoring is complete, the apparatus performs “auto-unloading” and sorting of final negative and final positive bottles.

### BACKGROUND

The presence of biologically active agents such as bacteria in a patient's body fluid, especially blood, is generally determined using blood culture bottles. A small quantity of blood is injected through an enclosing rubber septum into a sterile bottle containing a culture medium, and the bottle is then incubated at about 35° C. and monitored for microorganism growth.

Since it is of utmost importance to learn if a patient has a bacterial infection, hospitals and laboratories have automated apparatus that can process many blood culture bottles simultaneously. One example of such an apparatus is the BD BACTEC™ system, which is manufactured and sold by Becton, Dickinson and Co. U.S. Pat. No. 5,817,508 to Berndt et al. describes a prior art blood culture apparatus and is incorporated by reference herein. Additional descriptions of Blood Culture Apparatus are provided in U.S. Pat. No. 5,516,692 (“Compact Blood Culture Apparatus”) and U.S. Pat. No. 5,498,543 (“Sub-Compact Blood Culture Apparatus”) both of which are incorporated by reference herein.

Referring to FIG. 1, a culture medium and blood specimen mixture 22 are introduced into sealable glass bottles 1 that include optical chemical sensing means 20 on their inner bottom surface 21. Optical chemical sensing means 20 emanates differing quantities of light depending upon the amount of a gas in bottle 1. For example, the gas being detected by optical sensing means 20 can be carbon dioxide, oxygen or any gas that increases or decreases depending upon the presence or absence of microorganism growth in bottle 1.

As illustrated in FIGS. 1 and 2, a plurality of such bottles 1 are arranged radially on a rotating bell-shaped drum 2 within an incubator 5 in such a way that the bottoms of bottles 1 are oriented towards a drum axis 28. Bell-shaped drum 2 is hollow and is supported by a shaft 24 rotatably supported on one end by two large ball-bearings 3 and 4 mounted to a first side 51 of an instrument mainframe 50. In order to read information coming from each optical chemical sensing means 20 within bottles 1, a linear array of sensor stations 12 is mounted within rotating bell-shaped drum 2 to a second side 52 of instrument mainframe 50 at such a distance inside bell-shaped drum 2 that, during rotation of drum 2, individual bottles 1 are passing by respective sensor stations 15 in array 12. Each sensor station 15 of the linear array of sensor stations 12 comprises an excitation light source 11 and a collection end of an optical fiber 14.

Axis 28 of the bell-shaped drum 2 is oriented horizontally and parallel to a door 13, shown in FIG. 2, located on a front face of incubator 5. Horizontal orientation of axis 28 provides maximum agitation of the liquid culture medium and specimen mixture 22 and the gas within each bottle 1. During a load or unload operation, door 13 is opened which allows to access approximately one third of all bottles 1 simultaneously. Then, drum 2 is rotated until the next third of bottles 1 becomes accessible. In three steps, all bottles 1 are accessible.

Alternatively, axis 28 of bell-shaped drum 2 is oriented vertically with a slight tilting of approximately 20 degrees away from door 13. By adjusting the tilt angle, the degree of agitation can be modified, if required, for maintaining optimum growth conditions.

In operation, bell-shaped drum 2 is rotated by motor 6 and a belt 7. A circular member 8 and a sensor 9 form an angular encoder that provides information about which row of bottles 1 is passing sensor station array 12. Preferably, motor 6 is a stepper motor, allowing drum 2 to rotate either in a continuous mode or to stop drum 2 at appropriate angles to read from sensing means 20 within bottles 1 in a steady-state mode. The whole system is controlled by a control system 10 located inside rotating drum 2. Output ends of all optical fibers 14 of the linear array of sensor stations 12 are fed to one common photodetector (not shown) in control system 10 such that only one excitation light source 11 needs to be turned on at a time. Therefore, the control system “knows” from which sensing station 15 and, therefore, which bottle 1 the sensor light is being collected.

The apparatus shown in FIGS. 1 and 2 contains ten segments of blood culture bottles 1 with thirty-six bottles 1 per segment. Consequently, the total capacity is 360 bottles. The arrangement of bottles 1 on drum 2 allows for a relatively high packaging density, but improvement in density is still sought.

### BRIEF SUMMARY

Described herein is an apparatus for storing and monitoring blood culture bottles. The apparatus has a moveable rack configured as a drum having a plurality of receptacles therein for receiving blood culture bottles. The drum is disposed in a housing. The housing includes a heater and a blower for incubating the blood culture bottles at elevated temperatures. The housing may include other features such as vents, baffles, dampers etc. to further modulate and control the temperature and the temperature profile within the housing. Optionally the apparatus has a plurality of drums, each having a plurality of receptacles for receiving blood culture bottles.

Described herein is an apparatus for storing and monitoring blood culture bottles that has a drum-shaped rack having an exterior perimeter and an interior perimeter, the exterior perimeter having a diameter in excess of a diameter of the interior perimeter, the drum having a plurality of receptacles, the receptacles having a proximal end at the exterior perimeter and a distal end at the interior perimeter, each receptacle configured to receive a blood culture bottle, the blood culture bottle comprising a bottom portion and a neck portion. The bottle may be received by the receptacle such that either the bottom portion is received at the distal end of the receptacle, or the neck portion is received at the distal end of the receptacle. In one aspect the drum perimeters are disposed about an axis of rotation of the drum. In any of the above aspects, the plurality of receptacles is arranged in the drum as an array of receptacles, the array having receptacles disposed both vertically and horizontally, vertically aligned receptacles forming a column and the horizontally aligned receptacles forming a row. The apparatus also may have a bottle status indicator board removably attached to the drum-shaped rack interior perimeter, the bottle status indication board having a plurality of bottle presence sensors and a plurality of bottle status indicator lights that form an array, the array configured such that a bottle presence sensor aligns with a receptacle such that the presence or absence of a bottle is detectable by the bottle presence sensors. A status indicator light is positioned opposite a light pipe associated with a receptacle so that the light pipe is illuminated by the status indicator light to indicate a status of a bottle in the receptacle. The status reflects if the receptacle contains a blood culture bottle that is positive or negative for microbial growth wherein. In one aspect, the drum defines an interior space within the interior perimeter wherein at least a portion of drum electronics in communication with the sensors and detectors for interrogating the blood culture bottles are disposed in the interior perimeter of the drum.

In a further aspect, a motor is positioned within the interior perimeter of the drum-shaped rack. In a further aspect, the drum shaped rack may have a plurality of sections, each section of the drum-shaped rack having a plurality of columns of receptacles and a plurality of rows of receptacles, wherein at least one section is removable from a frame of the drum-shaped rack. In a further aspect, the bottle status indicator board is removably coupled to the frame behind a section. Further, a number of rows of the bottle presence sensors and a number of rows of the bottle status indicator lights in the bottle status indication board correspond with a number of rows of the receptacles in the section of the drum-shaped rack.

In a further aspect, the drum is vertically movable relative to the motor. In this aspect, the drum has a lifter for raising the drum relative to the motor. The apparatus may also have a locking tool for supporting the drum on the frame when the drum is in a raised position. In any of the above aspects, the apparatus further comprises a measurement board comprising sensors for determining the status of the bottle as the container is moved past the measurement board. In such aspect, the status of the bottle is one of positive for microbial growth or negative for microbial growth. In such aspect, the measurement board may be placed adjacent the exterior perimeter of the drum-shaped rack. In such aspect, the measurement board may have an alignment sensor. One example of an alignment sensor is a magnetic proximity sensor (aka Hall Effect sensor) placed adjacent to the measurement board or incorporated as a component the circuit board. Such a sensor would detect a magnet that is attached to the drum close to the first columns of bottles, often called the home position. A second. Another example of an alignment sensor may be an optical detector. In a further aspect, the alignment sensor is configured to detect flags positioned on panels that define each section of the drum-shaped rack. In one aspect, the flags are flanges extending from the panels that will pass through an optical beam of the optical detector. The interruption of the optical beam of the detector indicates that the flag has passed through the optical detector. In another example, alignment of the drum to the status indication board is done through monitoring of the readings from the optical sensors on the status indication board itself, which may be programmable and provide a digital reading of a proximity to an object. This digital reading may be used to sense the edge of a bottle column station, which, when correlated with a drum motor encoder value forms the basis for a drum to status indication board auto-alignment feature.

In one aspect, the flags are alignment flags and section flags. Each section of the drum shaped rack has a different flag distribution. In one aspect, alignment is indicated when the alignment flags interrupt the optical beam in a plurality of the alignment sensors. In a further aspect, the section of the drum-shaped rack is determined when alignment is indicated. In one aspect, a detected pattern of section flags indicates the drum section aligned with a door of a housing for the drum-shaped rack.

### BRIEF DESCRIPTION OF THE DRAWINGS

FIG. 1 shows a front-view of the interior of a blood culture apparatus for the detection of microorganisms of the prior art;

FIG. 2 shows a side-view of the interior of a blood culture apparatus of the prior art;

FIG. 3A and FIG. 3B illustrate perspective views of a blood culture apparatus housing for the module described herein;

FIG. 4 is a top view of an incubation and measurement module according to one embodiment of the present invention;

FIG. 5A-FIG. 5C illustrate the bottle rack drum with and without a section removed to reveal the BDSI board, in side views and a partial top view;

FIG. 6A-FIG. 6E illustrate the lift mechanism for servicing the motor in the drum interior;

FIG. 7A-FIG. 7E illustrate the BDSI board and measurement board and the controller board connection forming part of the measurement board and the alignment flags and section identifier sensor flags that work with the alignment sensors and the section identifier sensors on the controller board;

FIG. 8 illustrates the configuration of the alignment flags, and the section identifier flags that indicate to the BDSI board what drum section is aligned with a door to the drum module;

FIG. 9 illustrates one example of the progression of a drum/bottle status panel light patterns used to direct a user to align the drum to the BDSI board;

FIG. 10A-FIG. 10J illustrate aspects of the light sources and detectors for bottle interrogation;

FIG. 11 is the drum illustrated in FIG. 5 with transparent internally reflective light pipes forming receptacles for receiving the blood culture bottles;

FIG. 12A-FIG. 12C illustrate a light pipe used in the drum of FIG. 11;

FIG. 13 illustrates top view of the bottle drum as describe herein;

FIG. 14 illustrates a bottle in a receptacle with a light pipe;

FIG. 15A-FIG. 15E illustrate a bottle retained in the receptacle with the light pipe;

FIG. 16 illustrates one aspect of the drum described herein that illustrates the incremental number of degrees that the drum might be advanced to achieve alignment of one rack column of bottles with the bottle sensor;

FIG. 17 is an open front view of a blood culture incubation module adjacent to a module for loading and unloading blood culture bottles from the blood culture incubation module, according to one aspect of the apparatus described herein;

FIG. 18 is a view of the heater/blower assembly and the molded foam insert heat distribution system according to one aspect of the present invention;

FIG. 19 is an exploded view of the molded foam insert heat distribution system of FIG. 18;

FIG. 20 is a side cutaway view of the heater/blower assembly and the molded foam insert heat distribution system of FIG. 18;

FIG. 21 is a front view of a heater/blower assembly of FIG. 18;

FIG. 22 is a perspective view of the upper heater/blower assembly of FIG. 18 as it is placed in a cabinet of the blood culture incubation module of FIG. 17, according to one aspect of the invention;

FIG. 23 is a perspective view of the lower heater/blower assembly of FIG. 18 as it is placed in a cabinet of the blood culture incubation module of FIG. 17, according to one aspect of the invention;

FIG. 24 is a top-down view of the molded foam insert heat distribution system; and

FIG. 25 is a perspective view of the frame of the blood culture incubation module with the molded foam insert heat distribution placed therein.

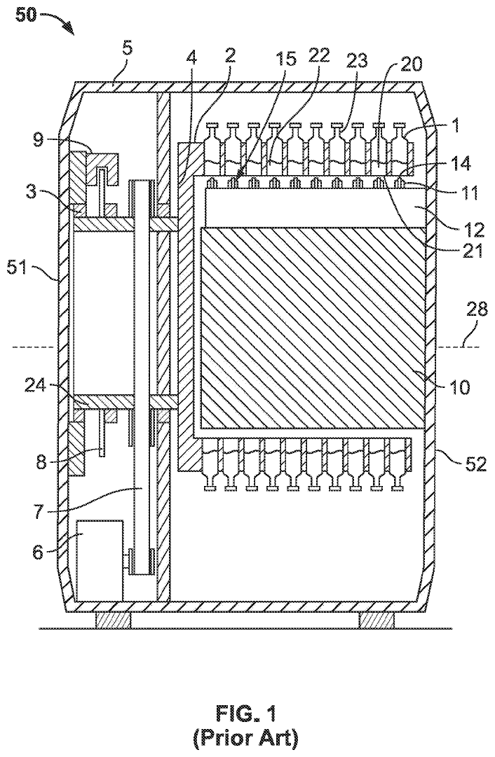

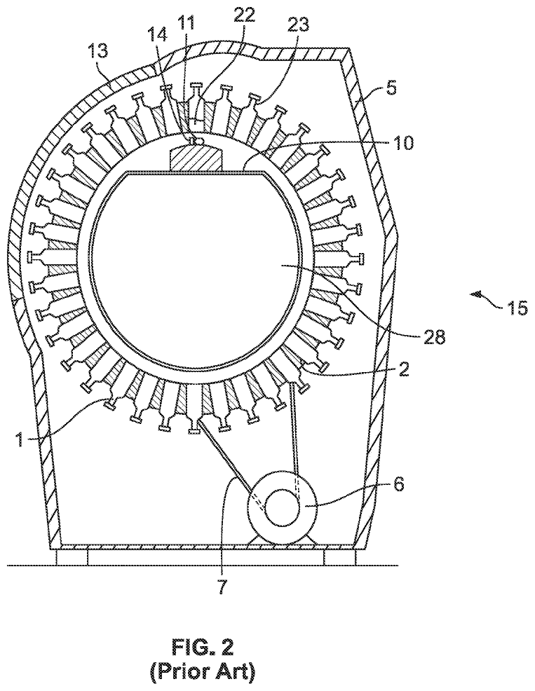

### DETAILED DESCRIPTION

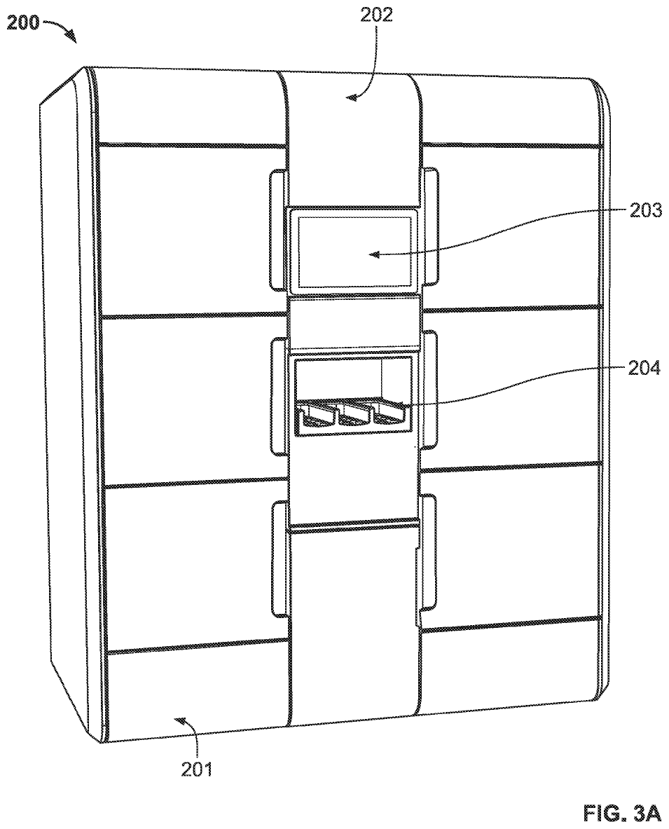

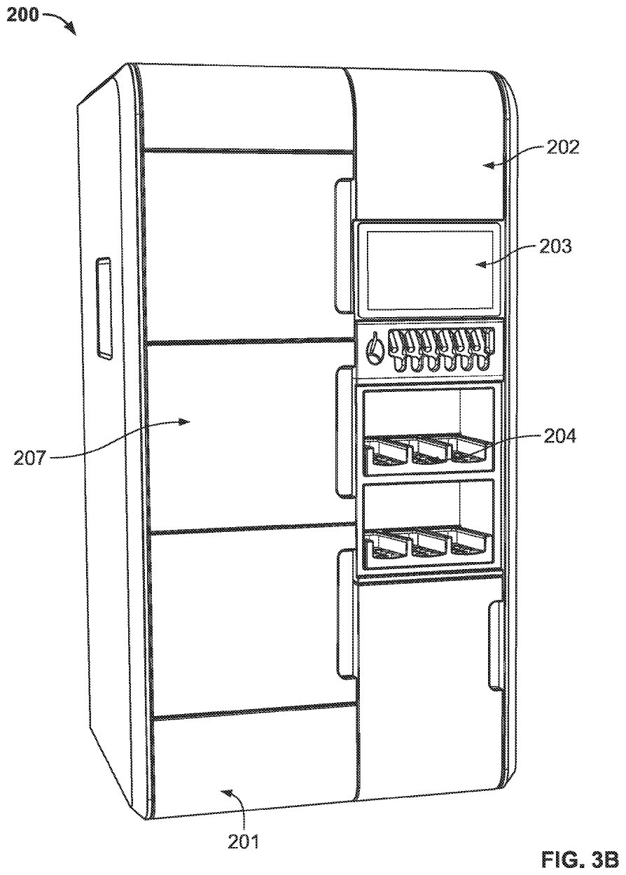

Described herein is a blood culture apparatus configured as an incubation and measurement module that, optionally, can be integrated with a larger end to end solution for processing biological samples to determine if such samples contain are contaminated with or infected by microorganisms. The module described herein can be placed in a cabinet such as is illustrated in FIGS. 3A and 3B. The cabinet 200 can provide power to the module, provide a controlled thermal environment to the module and a communication channel to the module. FIG. 3A illustrates a cabinet 200 with two three-door panels 201 that provide access to three bottle drums on each side of a central panel 202. Central panel 202 has touch screen 203 for data entry and used control. Central panel 202 also has a central station 204 for culture bottle input/output. FIG. 3B illustrates a cabinet with only one three-door panel 201. FIG. 3C illustrates a cabinet 200 with two two-door panels and 3D illustrates a cabinet that has only one two-door panel.

Then number of doors will depend upon the number of drums. The number of drum racks in a module is largely a matter of design choice, with one-, two-, and three-drum configurations being contemplated herein. The apparatus described herein is not limited to any specific number of drums.

The module has a high-density bottle drum. High density, as used herein is a description of drum configurations that allow culture bottles to be placed closer to each other to allow a greater number of bottles to be fitted into the drum compared to the prior art. The module is configured to align bottles with a limited number of reader stations. That is, the number of reader stations is less than the number of bottle receptacles in the drum. Optionally the drum is operated by a direct drive motor that can cause accelerated and decelerated drum movement (i.e., a rocking movement, intermittent rotation, etc.). A heater and blower are provided within the drum housing, or in spaced above or below the portions of the housing that receive the drums therein. The heater and blower circulate warm air around the drum. Optionally, the heater and blower will be configured to keep the temperature of the contents of all culture bottles in the drum within a predetermined narrow range of a specific target temperature. The predetermined narrow range is ±0.5° C. of the target temperature. The specific target temperature is in the range of 30° C. to 40°. Optionally, the target temperature is 36.55° C. Greater temperature uniformity will permit an increase in set point as there is less risk of “over-heating” samples. A greater temperature uniformity at higher temperature will therefore permit a faster time to detection. The motor will permit the drum to be positioned such that the user or the automated apparatus can access any bottle carried by the drum. When the sample in a bottle is determined to be positive for microbial growth, a workflow is activated to retrieve that culture bottle from the module. The module is configured to assist with that workflow. The placement of module components such as the blower in the module are largely a matter of design choice, and not described in detail herein. The module may include other features such as vents, baffles, dampers etc. to further modulate and control the temperature and the temperature profile within the module.

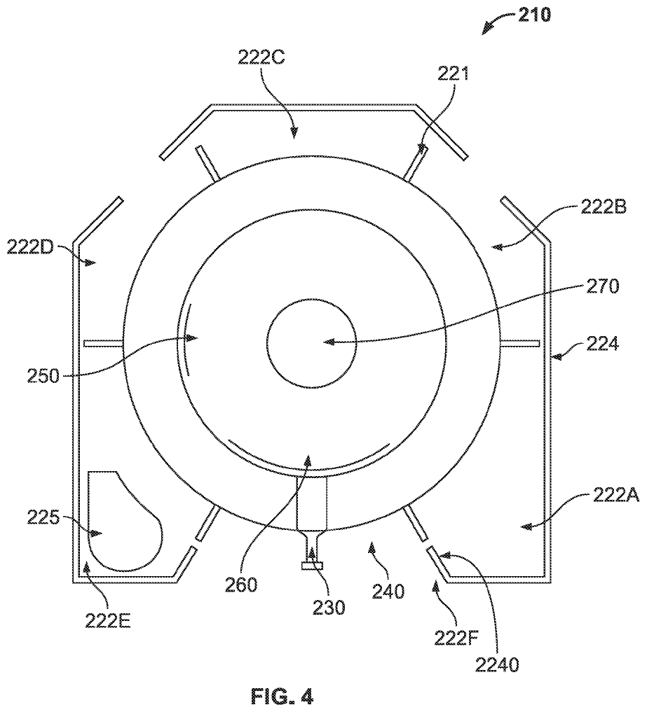

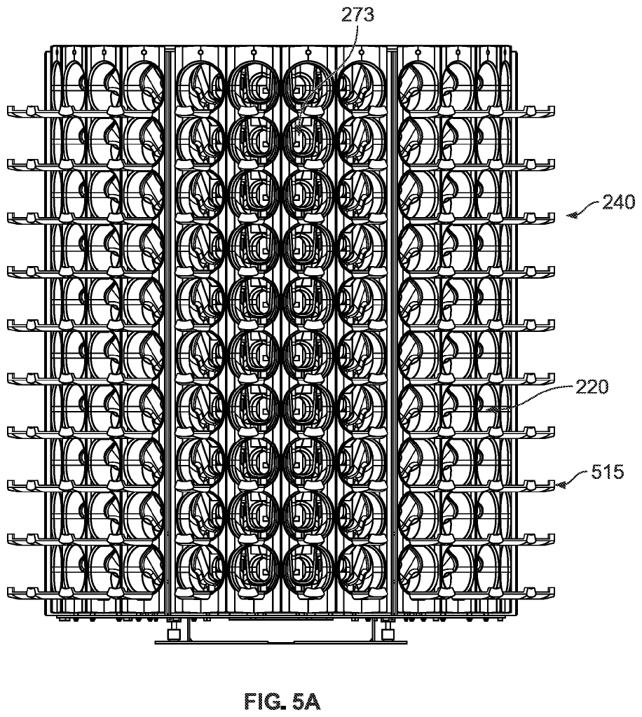

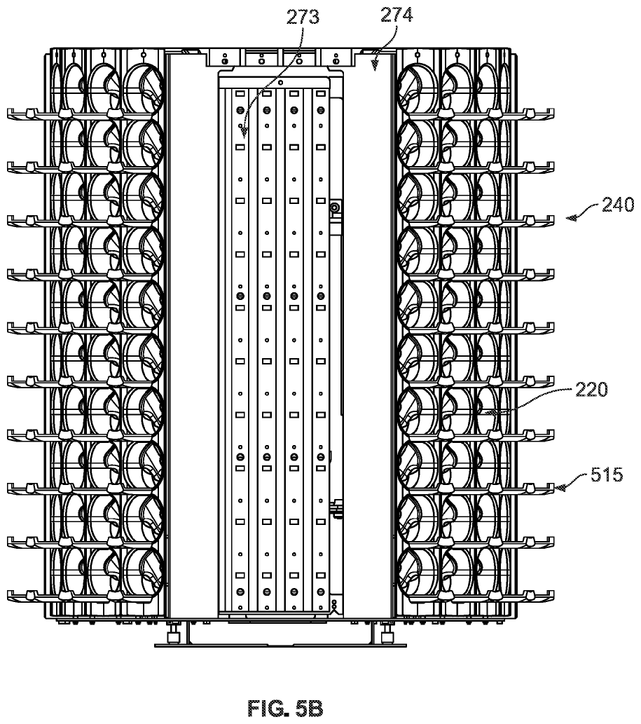

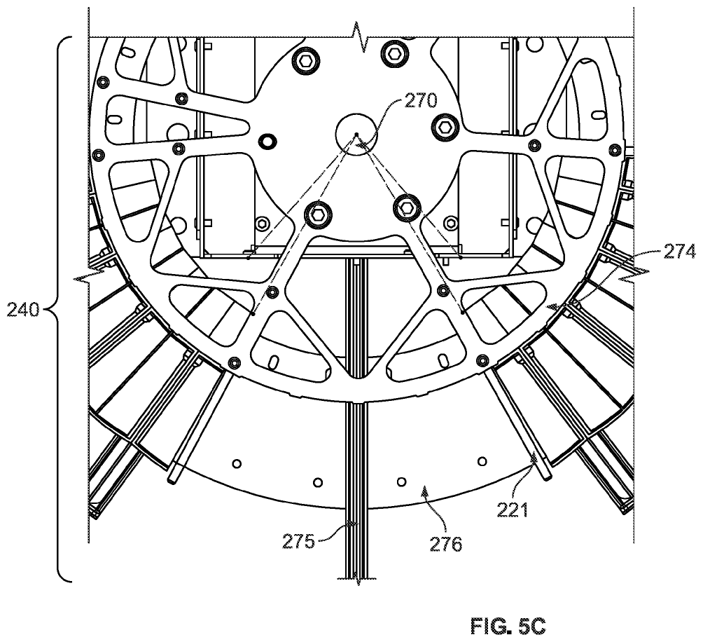

The module is configured to have LEDs and light pipes to indicate positive culture bottles to the user. Referring to FIG. 4, there is illustrated a top down view of an optional configuration of the module described herein. The module 210 has a housing 224, a blower and heater 225 for keeping the bottles 230 warm and a drum 240 with receptacles that hold the culture bottles. In the illustrated aspect, positioned in the interior of the drum are bottle presence sensor electronics 250 and culture bottle presence/status indicator electronics 260 (the BDSI board collectively includes these electronic components). A drive motor 270 is provided to rotate the drum 240. In other aspects described herein, the electronics are positioned adjacent the exterior of the drum. The drum 240 housing 224 has six panels 221 that define six drum sectors (222A-222F). As illustrated, about one-sixth of the drum contents (assuming the drum is full) are available for access at any given time since the span of one sector is about the same as the span of the opening in the housing through which bottles are added to or removed from the drum 240. FIG. 5A is a side view of the drum in FIG. 4. The culture bottles (not shown) are disposed neck inward in receptacles 220 in the drum 240 and received by cradles configured as a light pipe 515. The motor 270 is a direct drive motor provides high torque, little to no hysteresis, low noise, reliability and simplicity.

In one aspect, the drum 240 is configured such that the motor assembly 270 and the gearbox 271 are located in the interior of the drum 240. The drum 240 with receptacles 220 for receiving culture bottles (the culture bottles are not shown) is illustrated in FIG. 5A. As noted above, the drum 240 is assembled in sections of receptacles 220 and the drum 240 (with one section of receptacles 220 removed therefrom) is illustrated in FIG. 5B. A status indication board 273 that is more visible when the panel is removed is used to illuminate the light pipes 515 to indicate the status of the bottle cradled in the light pipe 515. The status indication board (BDSI board) 273 is also detachable and removable from the frame 274 of the drum 240. “Status” as used herein is the state of the blood culture bottle as determined by the module 210. The state of the blood culture bottle may be positive for microbial growth or negative for microbial growth. In one aspect, the status of a blood culture bottle is communicated by colored lighting of the bottle receptacle in the drum, with, in one aspect, green light indicating a bottle negative for microbial growth and red light indicating a bottle positive for microbial growth.

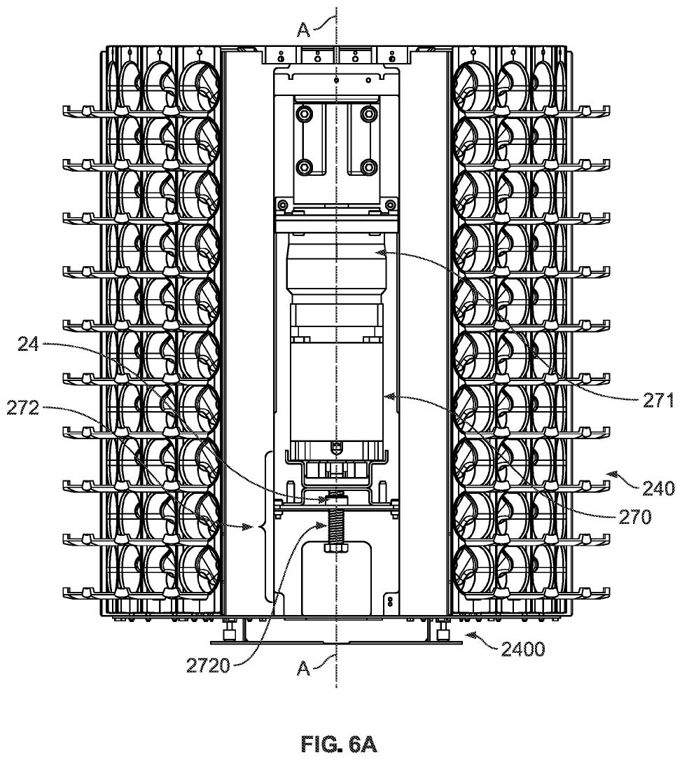

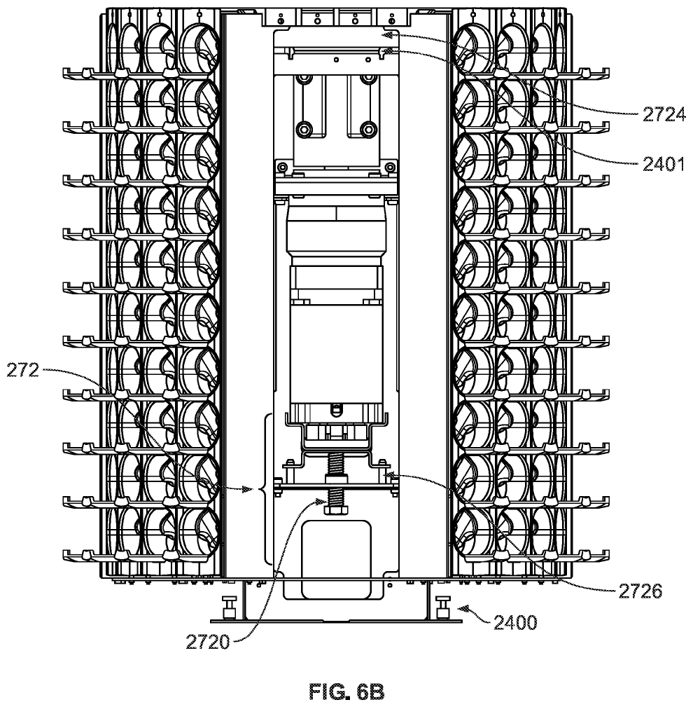

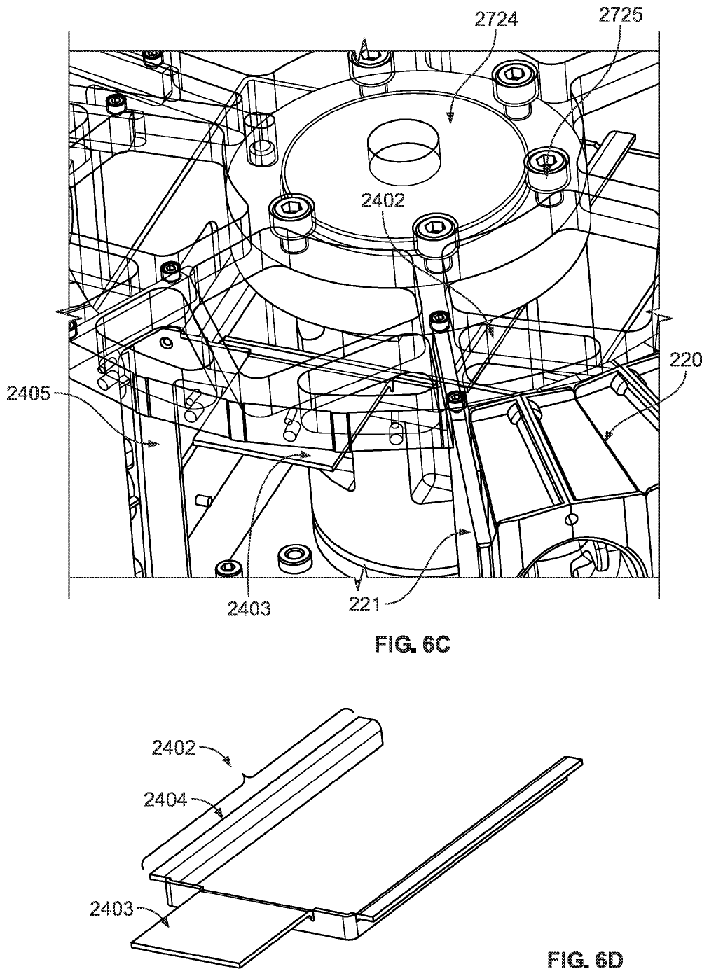

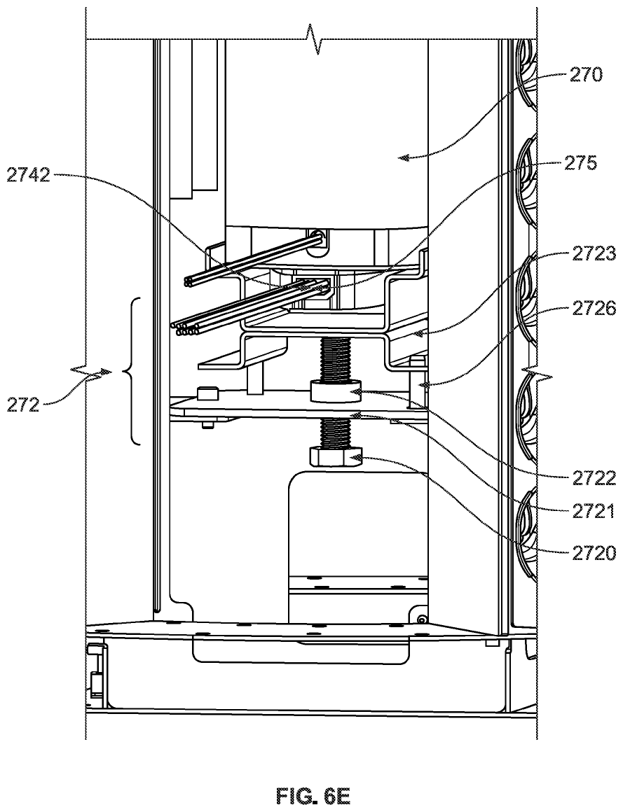

Referring to FIG. 5C, there is provided a top view of the frame 274 with the status indication board section removed therefrom. Cables 275 provide power to the motor assembly 270. Also illustrated is the bottom 276 of the frame 274. The drive motor assembly 270 drives the drum 240 to rotate axially. Bearings inside the gearbox of the drive motor assembly 270 provide both axial alignment of the drum 240 but also the requisite thrust load support required to advance the drum carrying a significant number of bottles 230. Referring to FIG. 6A, the axis A-A of the drive motor assembly 270 is aligned with the axis A-A of the drum 240. Consequently, the center of gravity of the drum 240/drive motor 270 assembly is on the center axis. The drum 240/drive motor assembly 270 is provided with a lifting mechanism 272 to expose the motor assembly 270 for service by lifting the drum 240. In one aspect, illustrated in FIG. 6E, the lifting mechanism is a screw 2720 that advances through motor assembly support plate 2721 and guide nut 2722. When advanced upward, screw 2720 forces plate 2723 upward. Plate 2723 travels upward along guide pins 2726. This raises the drum 240 off of drum supports 2400 and creates a lift space 2401 between the drum frame 274 and the rotor 2724 of the motor assembly 270. Referring to FIG. 6C, a locking tool 2402 is provided and is insertable into the lift space 2401 to lock the drum in place relative to the motor assembly 270 so that the motor can be serviced without carrying the weight of the drum 240. One example of a suitable locking tool 2402 is illustrated in FIG. 6D. The locking tool 2402, as illustrated has a handle 2403, affixed to a support bracket 2404. The locking tool 2402 is inserted between the rotor 2724 and the drum frame 274 (illustrated in phantom). The weight of the rotor 2724 and the drum frame 274 that is fastened to rotor via hex bolts 2725 is supported by service frame 2405 while the motor assembly 270 is being serviced. A detail view of the lifting assembly 272 is provided in FIG. 6E. Cables 275 provide power to the motor assembly 270. As explained previously, screw 2720 is advanced upward through plate 2721 and guide nut 2722 to raise support bracket 2723 (that travels along guide pins 2726) to lift the weight of the drum 240 off of the motor assembly 270 for service.

As previously noted, the apparatus is provided with a status indication board 273. As explained above with reference to FIG. 5A and FIG. 5B, the drum 240 is arranged in columns of receptacles 220, separated into sections, with multiple columns (e.g., four) in each section. In one aspect, one column in one section is reserved for reference bottles. User access to an individual section in the drum will be via a door that will give access to a single section (one of 222A-222F of receptacles 220) in the drum 240. The drum divided into sections is illustrated in FIG. 4.

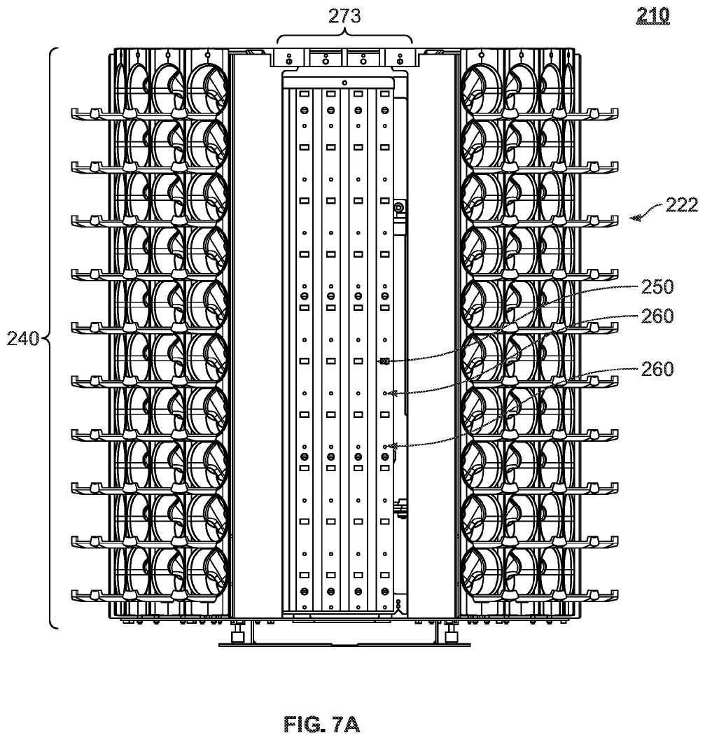

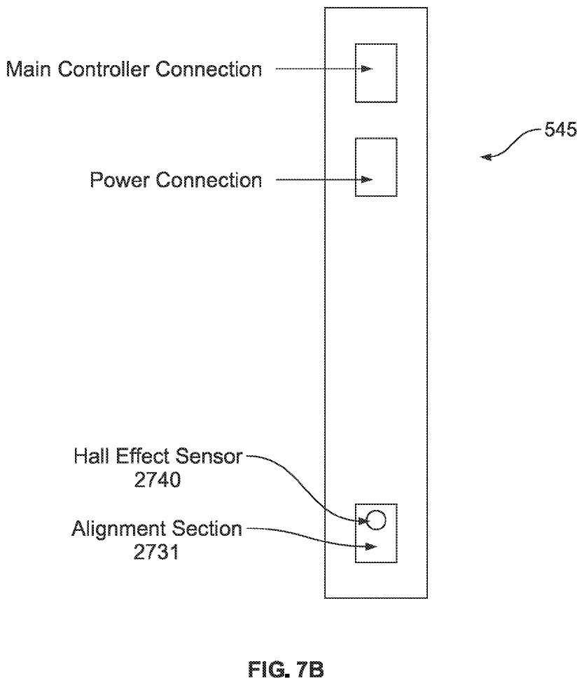

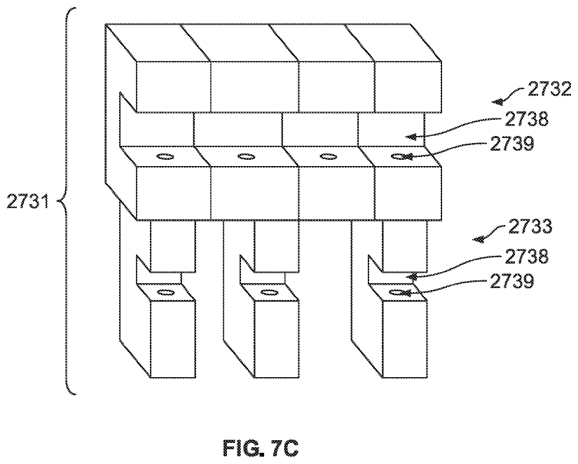

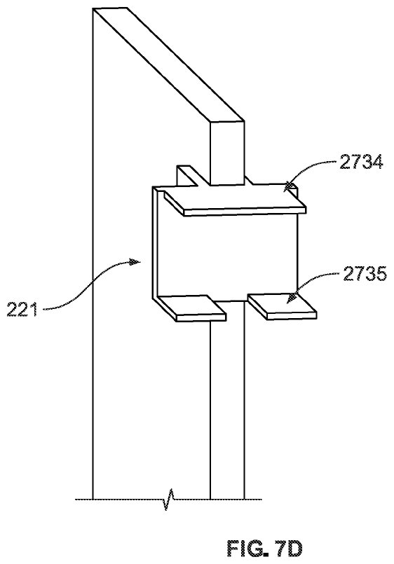

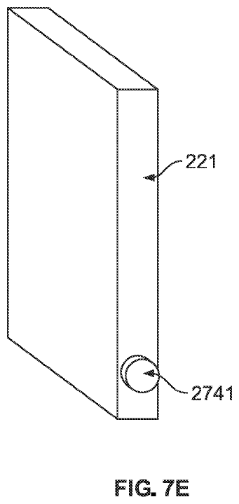

To sense bottle entry and removal and provide feedback to the user during the user's access to sections 222A-222F in the drum 240, there are bottle presence sensor 250 (bottle sensors) and culture bottle/status indicator electronics 260 (e.g., lights) on the status indication board 273. See FIG. 7A. The bottle sensors 250 each sense when a bottle is inserted into or removed from receptacle 220 in the section 222A-222F that is accessible through the door to the drum 240. The indicator lights illuminate light pipes 515 in the drum receptacles 220, which transmit the light to be visible to a user viewing the receptacles through the open door. This requires that the light pipes 515 of the receptacles 220 be aligned with the culture bottle/status indicator electronics 260 in the status indication board 273. When aligned properly, the module 210 may positively indicate the status of the bottles (e.g., positive for microbial growth or negative for no microbial growth), and to detect when a bottle is inserted or removed.

To access all bottles in the drum 240, a user must cause the module 210 to rotate the drum. As each section of the drum 240 is turned into view through the opened door, the user may have a way to ensure that the exposed section (one of 222A-222F) of the drum 240 is aligned with the bottle presence sensors 250 and status indicator lights 260 of the status indication board 273. With reference to FIG. 7A, in one aspect, the module 210 may have an alignment mechanism (located on measurement board 545 or the BDSI 273) that serves to align the receptacles 220 in the drum 240 with the bottle presence sensors 250 and the status indicator lights 260 provided on the status indication board 273. One skilled in the art will appreciate that alignment can be accomplished in many different ways, and that the alight mechanism is but one example of sensors that can be used to align the drum 240 with the module door and hence achieve alignment of the bottle receptacles with the bottle presence sensors 250 and the status indicator lights 260. Such alignment can be manual (e.g., the operator aligning the drum panels with the door opening); semi-automatic (a mechanism that the operator can control to advance the drum incrementally until alignment is achieved) or automatic (fiducials (e.g., the alignment flags)) that are detected and based on their detected location, aligning the drum with the fiducial(s).

In one aspect, the alignment mechanism also includes alignment flags and section identification flags attached to the drum panels that separate the drum sections. A measurement board or BDSI may include an alignment section with optical sensors that detect the flags on the drum panels as they pass by the optical sensors as the drum is rotating is provided on measurement board 545. See FIG. 5B. BDSI 273 is positioned adjacent the drum interior, so that it can detect flags on the drum panels 221. The optical sensors are in communication with a main controller that, in one aspect of the module described herein, inform the user/operator of the drum section that can be accessed through the open door.

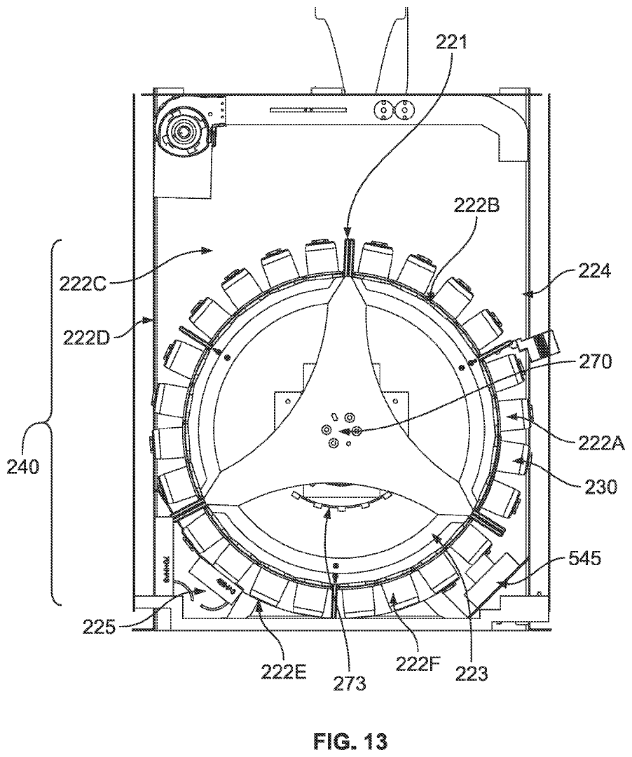

The status indication board 273 is also referred to as the bottle detection and status indication (BDSI) board 273. Status indication board 273 may be located behind the drum and aligned with the door opening as illustrated in FIG. 7A. As noted above, the module may also have a measurement board 545. In one aspect, the measurement board 545 is a controller board illustrated in FIG. 7B. With reference to FIG. 13, in one aspect, the measurement board 545 is fixed adjacent the exterior of the drum 240.

In one aspect, each column of the status indication board 273 may be either a single board or multiple interconnected boards. In the illustrated aspect, the four columns of the status indication board are connected to each other with, e.g., a flexible ribbon cable (not shown). The measurement board 545 is connected to a main controller board (not shown) for system communications. As illustrated in FIG. 7A, the status indication board 273 is mounted on the drum frame interior. By contrast, the measurement board 545, which may include an alignment section, is located on the drum exterior so that the flags 2734, 2735 may be detected by sensors 2739 on the measurement board 545.

Referring to FIG. 7C, in one aspect, the optional alignment section, 2731, may have a row of alignment sensors 2732 and a row of section identifier sensors 2733. In one aspect, the alignment sensors and the section identifier sensors may all be simple optical switches on the measurement board 545. As illustrated in FIG. 7C, the top row may contain four alignment sensors 2732 and the bottom row may contain the section identifier sensors 2733. The alignment sensors 2732, and the section identifier sensors 2733 may each have a notch 2738 in which a sensor 2739 (e.g., an optical switch) is disposed. Adjacent sections of the drum 240 may be separated by vertical panels 221 (also referred to as a drum rib, or wall) that extend out from the outer surface of the drum. Such are illustrated in FIG. 4 and FIG. 6C. As illustrated in FIG. 4, the drum panels or ribs 221 on either side of a section (222A-222F) align with the fascia 2240 of the housing 224 for a clean look when the user has the door open. The drum ribs 221 also extend to the inner surface of the drum as illustrated in FIG. 5C.

Each drum rib 221 inside the drum will have multiple flags (2734, 2735), one flag 2734 for drum alignment, and another set of one or more flags 2735 below the drum alignment flag 2734 for section identification. Flags 2734 and 2735 are detected when they pass through the notch 2738 and in the respective alignment 2732 and section identifier sensors 2733.

As noted above, each panel or rib 221 carries two flags, an alignment flag and a section identifier flag. As can be seen in FIG. 7D, the alignment flag may be a continuous flag to span across all four of the alignment sensors 2732. When the drum rotates such that all four of the optical switches in the alignment sensor 2732 register the presence of the flag, this indicates that the drum is in alignment. In one aspect, the sensors have an optical beam that transmits through the sensor gap. When the flag enters the gap, the optical beam is interrupted and this interruption registers as an indication of the presence of the flag. Although the flag interrupts the signal, this is referred to as the “on” state of the sensor, since, in this state, the sensor detects the presence of the flag. The section flags 2735 are configured as a unique identifier of a particular section of the drum. Consequently, the size and number of section flags varies for each section, so that each section creates a signal unique to its particular section. As illustrated, the sensors 2739 are positioned in a respective notch 2738 through which the alignment flags/section identifier flags pass. The sensors are configured as an emitter/receiver pair, only one of which is illustrated in FIG. 7C. In normal operation, the signal from emitter to receiver is uninterrupted. When the flags pass through the sensors, they “break” the optical beam and this provides an indication that the flags are present in the sensors. For a positive indication of alignment, the alignment flag must break the optical pathway for all of the alignment sensors. At that precise point, the number of signals “broken” for the section sensors will indicate which section is about to be rotated into position. Referring to FIG. 8, the number of sensors “fired” by the section flags passing through identifier sensors may inform the control software which section is present at the sensor location. At the point when the alignment flag 2734 is fully activating all alignment sensors 2732, the section identifier flags 2735 have activated the section identifier flag sensors 2733 by a wide margin. This will ensure the correct section of the drum 240 is identified when the alignment is correct. As stated previously, the alignment flag is the same size and configuration for each section, while the section flags each has a unique configuration such that the signal produced by a section flag indicates a particular section of the drum. As noted above, the alignment mechanism described above is one example of a mechanism that may be used to align the drum. One skilled in the art is aware of other suitable mechanisms for achieving alignment of the drum with the door and other module electronics (e.g., the status indication board 273).

In another aspect, the drum may have drum motor encoder 2742 in communication with the measurement board 545. In this aspect, the alignment section 2731 includes a Hall Effect Sensor 2740. In one aspect, the Hall Effect Sensor is placed approximately in the center of the measurement board 545 as illustrated in FIG. 7B. The Hall Effect Sensor detects a flag that, in one aspect, is a magnet 2741. Referring to FIG. 7E, the magnet 2741 is positioned on the drum rib 221. This position is by way of example, but placing the magnet on the rib, rather than some other drum surface, gets the magnet closer to the Hall Effect Sensor and thereby provides a greater likelihood that the Magnet will trigger the Hall Effect Sensor as it passes by the Hall Effect Sensor. The magnet 2741 is detected by the Hall Effect Sensor in fly by fashion. When the magnet 2741 is detected, a signal is communicated to the Main Controller. Because, the Main Controller now has a starting position, the encoder 2742 can then be used to associate drum rotation with a target position of the drum in the cabinet. There are two objectives for alignment. The first objective is the alignment of the status indicator lights 260 in the BDSI 273 with light pipes 515 in the receptacles 220. The second objective is alignment of the drum ribs 221 with the fascia 2240 of the housing 224, such that only the portion of the drum between the drum fascia are visible when the door 207 is opened. Typically, if one objective is met, the other objective is also met, and the drum is aligned.

The drum encoder 2742 (FIG. 6E) associates shaft rotation with degrees of drum rotation. Since the signal produced when the magnet flag flies past the Hall Effect Sensor 2740 indicates an origin, the drum encoder may provide information to the Main Controller to determine the position of the drum, and the amount of shaft rotation required to move the drum from a first position to a second position. Although the position of the magnet on the drum perimeter is largely a matter of design choice (as long as the magnet will be detected by the Hall Effect Sensor as it passes by the Hall Effect Sensor), it is advantageous if the magnet is positioned at or near the column of the drum rack reserved for calibrator bottles. With reference to FIG. 13, there is top-down view of a drum 240 with twenty-four (24) columns. There are four bottle station columns in each of the six sections 222 A-F. In one aspect, the drum is modified to include a seventh section containing a single column. This seventh section may contain calibrator bottles that, in one aspect, have a fixed fluorescence. The number of columns in each section, and the total number of columns is indicated in FIG. 8. When the calibrator bottles are placed adjacent to the Measurement Board sensors 5451, the fluorescence induced by the light sources 5451 is detected by the Measurement and compared with the known fluorescence associated with the calibration bottles. If the measured fluorescence is within an accepted tolerance of the known fluorescence, the Measurement Board detectors are determined to be functioning correctly. It is adjacent to this seventh section that the magnet flag is located in one aspect of what is described herein. One skilled in the art will appreciate that the number of columns, the number of drums, the drum capacity and other such aspects are a matter of design choice and are described herein for purposes of illustration.

The drum motor encoder is rigidly coupled to the motor shaft 24 of the drive motor assembly 270. The drive motor shaft 24 is coupled to a gearbox 271 that is coupled to the bottle drum 240.

In one aspect, the main controller has a look up table of drum conditions that can cause the drum to be misaligned. The look up table provides drum conditions that cause the drum to be misaligned. For example, an alignment tolerance for the drum 240 may be determined by populating the drum 240 and recording loading arrangements that cause the drum 240 to go from an aligned state to an unaligned state. As used herein, the aligned state is when the bottle presence sensors 250 in the BDSI 273 are aligned with the bottles 230 so that a bottle 230 is detected by each bottle presence sensor 250 when the alignment sensors 2732 indicate that the drum is aligned. An unaligned state is when one or more of the bottle presence sensors 250 do not detect the presence of a bottle 230 in the bottle receptacle 220 when the alignment sensors 2732 indicate that the drum 240 is aligned. Boundary conditions are conditions that change the drum from an aligned state to an unaligned state. Those boundary conditions are noted and used to define the alignment tolerances. The drum is determined to be out of alignment when a bottle presence sensor 250 for a bottle receptacle 220 with a bottle 230 therein does not light up when the alignment sensors indicate that the drum is aligned. When this occurs, the operator knows that the drum 240 is out of alignment.

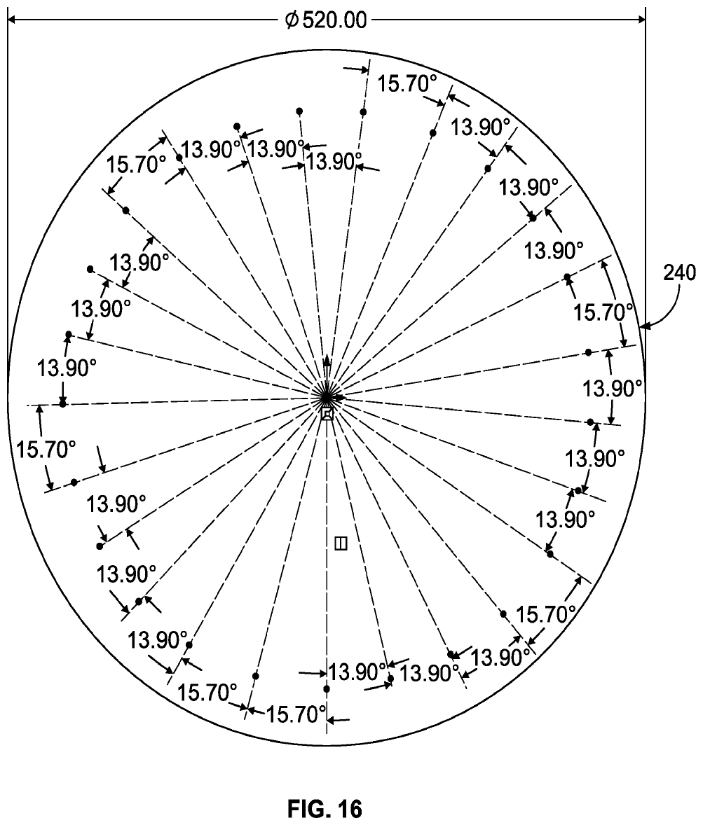

In another aspect, the drum motor encoder provides feedback to the operator when the operator is moving the drum manually. This is accomplished by controlling the movement of the drum 240 incrementally, so that the operator can determine when the bottle presence sensors all indicate the presence of bottles in the receptacles 220. As noted above, the drum encoder increments drum movement based on shaft rotation. In one aspect, an incremental drum revolution is selected to control how much the drum will rotate in response. With reference to FIG. 16, there illustrates one example of the degree range for incremental movement of the drum 240. FIG. 16 illustrates the degrees of rotation between the rack columns in one example. The degrees of rack rotation from one column to the next in one section is 13.9 degrees. The degrees of rack rotation from a column in one section to a column in a second section is 15.7 degrees. The motor encoder measures shaft rotation by increments, and based on the increments, the controller may determine the degrees of drum rotation. When the drum is rotated manually, the encoder may still increment based on shaft rotation and drum position/alignment may still be determined by the main controller based on the shaft increments communicated from the encoder to the main controller.

The above arrangement allows a module controller (e.g., measurement board 545 or BDSI 273) to manage drum alignment without requiring communication with the main controller. The measurement board 545 may handle the process of helping the user align the drum to the bottle detection and status indication board (BDSI) once the module door is opened. The module will display the status of the stations whenever proper alignment is determined/indicated as described above.

The Main Controller may also handle the alignment of the drum prior to the door opening so the bottle status for the drum section visible when the user opens the door can already be lit when the user opens the door. In one aspect, the Main Controller determines when a local controller (i.e., a controller in the module as opposed to the Main Controller) will activate the status lights on the status indication board 273. To commence a manual workflow, for example, the Main Controller may send a command to a module controller start alignment. From there, the Main Controller or the module controller may manage drum motion and status display. The local controller can be located anywhere in the module. In one aspect, the local controller is located on the measurement board 545. In another aspect the local controller may be located on the status indication board 273. The door to the module may be opened for a variety of reasons described herein. When the module door is closed, the status indicator lights are turned off. A command from the main controller may start alignment. When the door is to be opened, an alignment process commences, and the drum is advanced until the alignment flag activates all of the alignment sensors mounted on the measurement board 545. When alignment is achieved, the local controller may also transmit to the main controller the specific drum section aligned with the door determined by the section sensor and the section flags detected when the alignment flag has activated the alignment sensor.

In one aspect, the main controller, in cooperation with and based on information from the status indication board 273, provides a status map for the module 210. The status map is updated when the user manually enters and removes bottles from the drum, or when bottles are added or removed by an automated apparatus in communication with the module 210. When the door to the module is to be opened, the main controller shares the status map with the status indication board 273. When bottles are added or removed from the module 210, the map is updated and shared with the main controller. The main controller may share the information with the command center if the module is not operating in isolation mode.

In one aspect, the local controller will enter an error state and indicate to a user that the module door must be closed. The conditions that might cause an error state are largely a matter of design choice, but can be something like a drop off in module temperature, a misalignment, etc.

As illustrated in FIG. 7A, the status indication board is equipped with a plurality of lights that may convey information to the user. Although located behind the drum 240 and the bottle receptacles 220, the lights convey information to the user through the light pipes 515 in the receptacles 220. In one aspect, the status indication board 273 may convey information as a pattern of light/light of different colors. The light patterns/colors/meanings are a matter of design choice. Examples of the information conveyed include the station/receptacle status (blocked, available, etc.) and bottle status (positive, negative etc.). The status displayed is communicated from the main controller to the local controller.

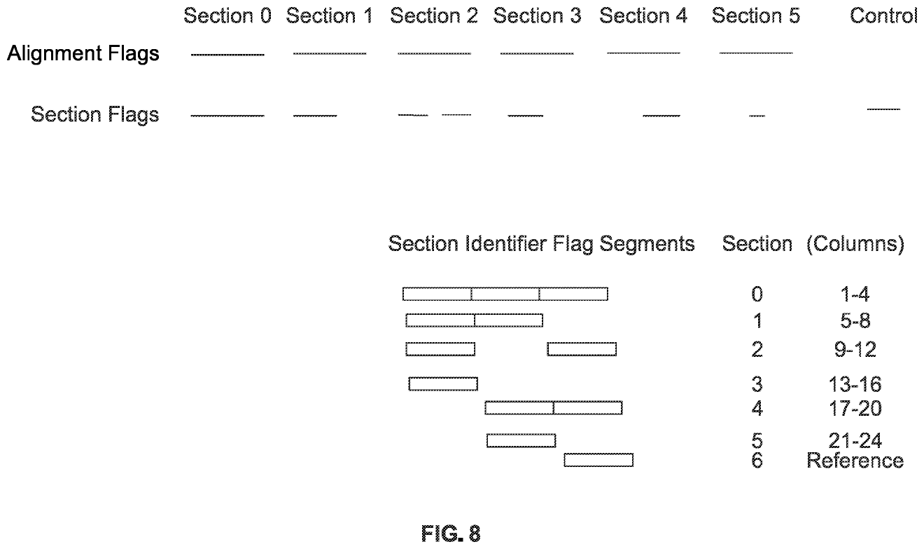

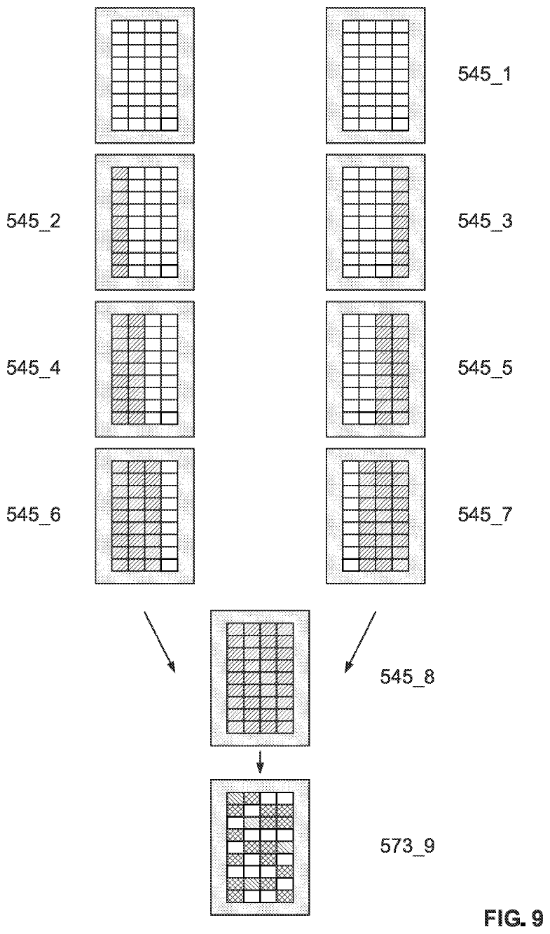

Measurement board 545 may be in communication with a panel that conveys alignment status to a user. FIG. 9 is one example of the progression of displayed alignment status with multiple lights for conveying information about the alignment status of the instrument. For example, when the alignment flags 221 are not in alignment with the alignment sensors 2731, the lights are all dark 545_1. As the drum section moves into alignment, the columns illuminate as the flags begin to activate the alignment sensors either moving from left to right (illuminating the left column 545_2 first) or from the right to the left (illuminating the right most column 545_3 first). This progression of the alignment flags through the alignment sensors (either from the left to the right or from the right to the left) are illustrated for detection of the alignment flag by the second alignment sensor (545_4; 545_5) or the third alignment sensor (545_6; 545_7). When all four alignment sensors detect the presence of the alignment flag, all alignment sensor indicator lights are illuminated (545_8). In another aspect, the panel will simply communicate no alignment (all lights off) or total alignment (e.g., all lights on).

Once all of the alignment sensors are lit, the status indicators change to the station statuses 273_9 and the user may begin manual operations such as placing bottles in or removing bottles from the module 210. The panel will still provide for an indication of alignment but will have some tolerance built in as the drum may move slightly during manual operations. This will avoid triggering a misalignment reading that might require a module reset. In one example, once complete alignment is achieved, alignment continues to be indicated as long as either the left most or right most alignment sensor continues to detect the presence of a flag. If there is no flag detected by any of the alignment sensors, then the local controller turns off all of the indicator lights and a new alignment protocol may be commenced.

Once alignment is confirmed, a panel 273_9 in communication with the status indication board 273 will indicate the status of the individual bottles in the receptacles. For example, two-way cross hatched lights indicate a sample negative for microbial growth (col. 1, row1, col. 2, rows 3 and 8, and col. 4, row 5). One-way cross hatched lights may indicate bottles positive for microbial growth (Col. 1, rows 2, 4, 6, 8 and 9; Col. 2, rows 1 and 5; Col. 3, rows 2, 3, 5, 6, and 8; and Col. 4,, rows, 2,, 7, and 9). The unlit lights indicate that no bottles are present at those locations. One advantage of the present design is that the user can advance the drum manually when the door is opened. To advance the drum automatically, it is advantageous to have the door closed so that nothing gets caught in the advancing drum. In one aspect, the drum may need to be powered off when the door is open. In that mode of operation, the drum may not be able to be advanced when the door is open.

In another aspect, rather than using light pipes to convey bottle status as described above in the context of FIG. 9, a BDSI panel may be provided on the exterior of the drum to convey the alignment information. The BDSI controller would be connected to the BDSI panel to illuminate the indicators, which could be light pipes, lenses etc.

As described above, the rotating drum rotates past the measurement board 545. (The alignment performed by the measurement board is the shark fin alignment, not the BDSI alignment) Since the drum 240 rotates past these various detection devices on the measurement board, the measurements occur in what is described as a “fly-by” fashion, fly-by being the rotating drum moving the bottles past the measurement electronics as the measurements are being made. The measurements being made ascertain whether the blood culture bottles are positive or negative for microbial growth. As such, measurement sensors on the measurement board 545 are provided to interrogate the bottles to determine if their internal gas composition or pH is dynamic (i.e., changing) in a manner that is indicative of metabolic activity inside the bottle as a consequence of microbial growth. For example, bottles with a measured increase in carbon dioxide or a measured decrease in oxygen concentration over time may be determined to be positive for the growth of microorganisms. To make this determination, light sensors are directed at a chemical sensor in the bottle that is indicative of bottle conditions (e.g., oxygen concentration, carbon dioxide concentration, pH). The location of the chemical sensor in the bottle will depend upon what the sensor is measuring. The headspace in the bottle is the portion of the bottle interior where the gasses are separated from the liquids and solids in the blood culture (i.e., the sample, the nutrients, etc.).

Since a culture bottle may be interrogated multiple times before a determination is made that the culture bottle is positive or negative for microbial growth, the measurement conditions must be consistent enough measurement to measurement, or an adjustment may need to be made to the measurement in the instance of distance variability. This means that the light from the interrogation sensors 2501 and the distance from the bottle sensor to the photodiode detector 2602 should remain relatively constant measurement to measurement.

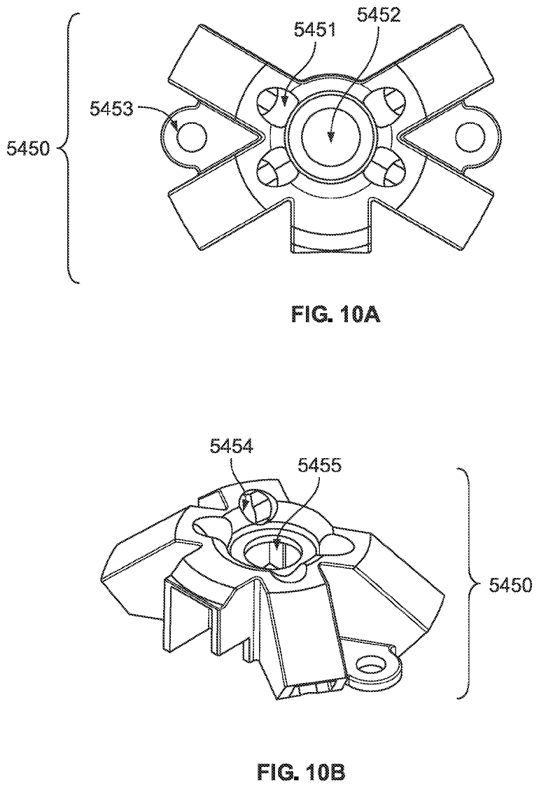

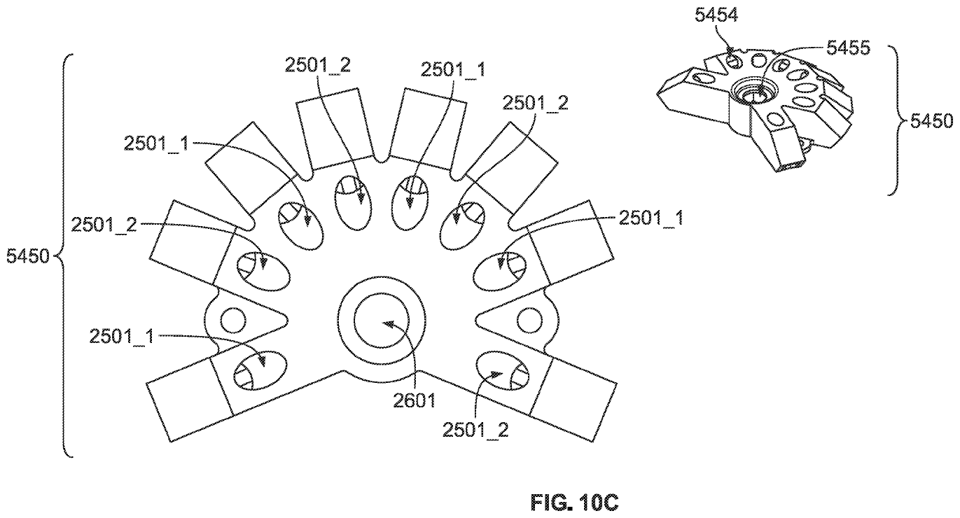

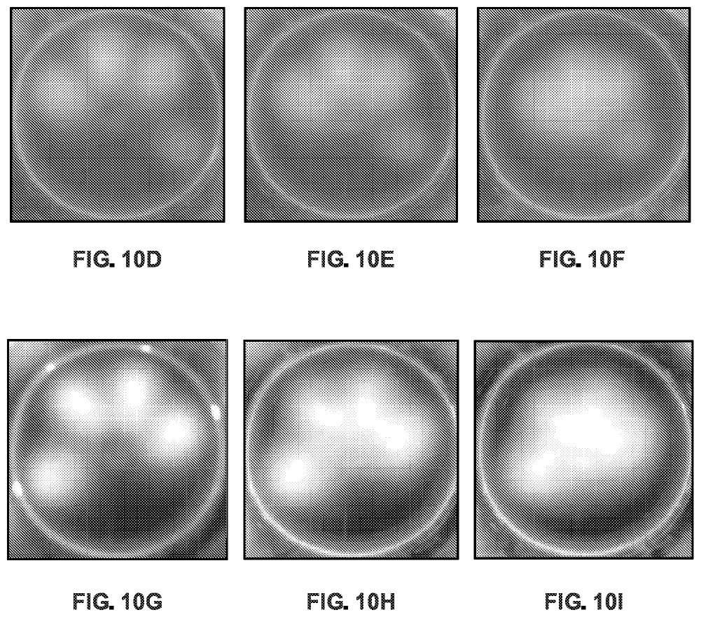

As described above, the bottles are interrogated on a column-by-column basis as the drum 240 with the rows of bottle receptacles 222 rotate past the measurement board 545. Referring to FIG. 10A-101, the sensors (e.g., the light sources 5451) and the detector 5452 are provided in a housing 5450 which is fastened to and extends from the measurement board 545. Light sources 5451 are positioned around a single light detector (photodiode 5452). The housing has a fastening mechanism (flanges 5453) for fastening the housing 5450 onto the measurement board 273. As illustrated in FIG. 10B, the housing 5450 has ports 5454, 5455 for receiving the light sources 5451 and for receiving the photodiode detector 5452. The ports 5454, 5455 are configured so that the light sources 5451 surround the photodiode detector 5452 and are angled towards the photodiode detector 5452 such that the light emitted by the light sources 5451 intersects above the photodiode detector 5452 on the bottle sensor (not shown) directly opposite the photodiode detector 5452.

As the distance between the bottle and measurement board 545 increases, the combination of the excitation light from the light sources 5451 spreading out, and the fluorescence it induces in the bottle sensor being farther from the photodiode detector 5452, cause the signal produced by the photodiode detector 5452 to decrease. The light sources 5451 may be located farther radially from the photodiode 5452 since the point of intersection of the light from the light sources 5451 relative to the bottle sensor is what provides measurement to measurement consistency. It is advantageous if the light emitted by the light sources 5451 intersects with the bottle behind the bottle sensor directly opposite the photodiode. The intersection point is far enough behind the bottle sensor, causing the illumination of the sensor to be off center such that the fluorescence induced by the light sources 5451 is partially out of the field of view of the photodiode 5452.

As the bottle moves away from the measurement board 545, the light from the light sources 5451 converges onto the center of the bottle sensor disposed in the blood culture bottle and therefore moves into the field of view of the detector 5452. This additional fluorescence of the sensor, caused by the additional light from the light sources impinging on the bottle sensor, offsets the decrease in fluorescence to be detected by the detector 5452 by virtue of the fact that the bottle, and the sensor in the bottle, are slightly further away from the detector 5452.

Referring to FIG. 10C, the housing 5450 has eight light sources 2501 therein. Four light sources are a first color and are designated 2501_1. The light sources are light emitting diodes (LEDs). In one aspect, four light sources are a second color and are designated 2501_2. In one aspect, the first color is green, and the second color is cyan. As illustrated, the color of the light sources alternates around the photodetector 2601. The housing 5450, with the ports 5454 for receiving the light sources 2501 and the ports 5455 for receiving the photodiode detector 2601, is illustrated in the upper right. FIGS. 10D-10I illustrate the transition of light impinging on the bottle sensor (from closer to farther). FIGS. 10D-10F illustrate this transition for the cyan LEDs and FIGS. 10G-10I illustrate the transition for the green LEDs (again, from closer to farther). The above design mitigates the measurement-to-measurement variations that arise due to a measurement-to-measurement difference in the distance between the light sources/photodetector and the bottle sensor. The intensity of a diffuse light source drops proportionally to the square of the distance from the light source. This is why the photodiode signal produced by the fluorescence that results for the light from the light sources drops as the distance between the measurement board 545 and the bottle increases. Not only does the fluorescence intensity received by the photodiode decrease, but the intensity of the source light impinging on the sensor also decreases.

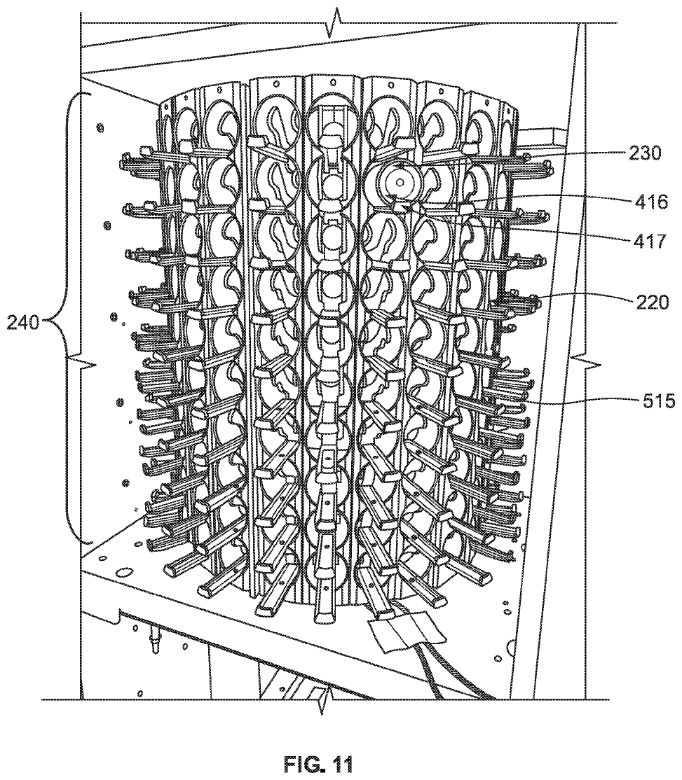

Referring to FIG. 11, the bottle drum 240 has receptacles 220 for receiving culture bottles disposed neck-in therein as described above. The receptacles 220 have a light pipe 515 formed in the bottom portion of the receptacle that also defines the bottom edge of the receptacles 220. The light pipes are formed from a material that will transmit light through the light pipe structure but prevent the light from emanating from the light pipe, to prevent appreciable cross-talk from an illuminated light pipe to an unilluminated light pipe. Examples of suitable material include polycarbonate (e.g., Makrolon 2258), and acrylic (e.g., polymethyl methacrylate). Makrolon® (formerly Hyzod®) is a trade name for Covestro (formerly Bayer MaterialSciences). These materials are all polycarbonate which is a very tough, high impact plastic material. Translucent materials are contemplated, but partially transparent light pipe materials may make perceiving the color of the light pipes more challenging.

Referring again to the status indication board 273, the LEDs that illuminate the light pipes 515 may be a plurality of LEDs that may illuminate the light pipe in a number of different colors, each indicating a different status of the blood culture bottle being held in the receptacle 220 that has the light pipe. In one aspect, the LEDs illuminating the light pipe are on the status indication board and about 5 mm from the light pipe. As described herein, once the culture bottle status is determined, the BDSI, in conjunction with the local controller or main controller, determines the color to illuminate the light pipe 515 (e.g., red for positive, green for negative, etc.). The light pipes are configured to both provide a color indication of bottle status and retain the culture bottle 230 in the drum receptacle 220. In this configuration, the culture bottle bottom 416 is secured in the receptacle by tab 417.

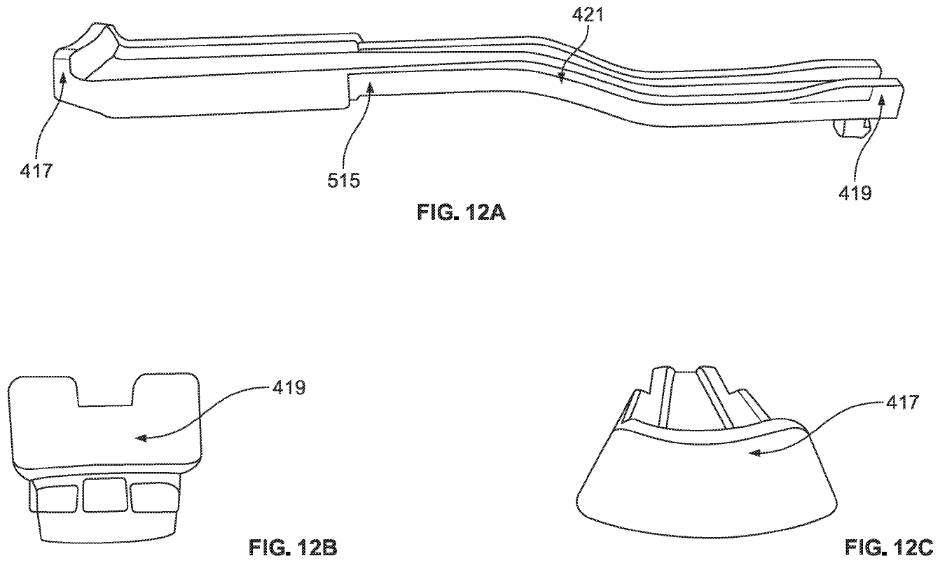

FIG. 12A is a perspective view of the light pipe receptacle 515 with the light entrance end 419 and the tab 417. The light pipe 515 is configured as a waveguide for the LEDs positioned at the light entrance end. As such, the light pipe is configured to have a total internal reflection to render it suitable as a waveguide. In one aspect, the light pipe 515 has a refractive index of about 1.52, which is higher than that of the surrounding air. In one aspect, light pipe is cladded and the refractive index of the waveguide portion of the light pipe 515 is higher than the refractive index of cladding on the light pipe 515. FIG. 12B is the light entrance end 419 and FIG. 12 C is the tab 417.

In order for the light pipe to have the requisite total internal reflection, any curves must be mild, with no sharp curves or dead corners. The mild curvature is illustrated as 421 in FIG. 12A by way of example. In one embodiment, the light pipe has a body length of about 110 mm from the light entrance end 419 to the tab 417. For purity of transmission, it is advantageous if the presence of foreign particles and bubbles is minimized. In one aspect, the light pipe is molded polyacrylate. While the use of 3D printing to form the light pipes is contemplated, it is easier to control the quality of light pipes formed by molding. In one aspect, the light pipe has very low, little or close to zero internal absorption, and is free of foreign particles and bubbles. For example, and not by way of limitation, low or little internal absorption is less than about 0.2 dB cm⁻¹.

Referring to FIG. 13, the bottles 230 (facing inward) in the drum 240 are placed into one of six sectors (222A-222F) in the module 210. The sectors are demarcated by vertical panels 221 that extend outwardly from the drum 240. The span between adjacent panels is approximately equal to the span of a door into the housing 224 to completely shield the user from the inside of the module when a section of culture bottles is being accessed by the user. The module 210 also includes a blower and heater 225 for keeping the bottles 230 warm. Positioned in the interior of the drum is the status indication board 273. A drive motor assembly 270 is provided to rotate the drum 240. The measurement board 545 is positioned on the outside of the drum and takes fly by measurements of the bottle and drum flags to determine bottle status and drum alignment, respectively.

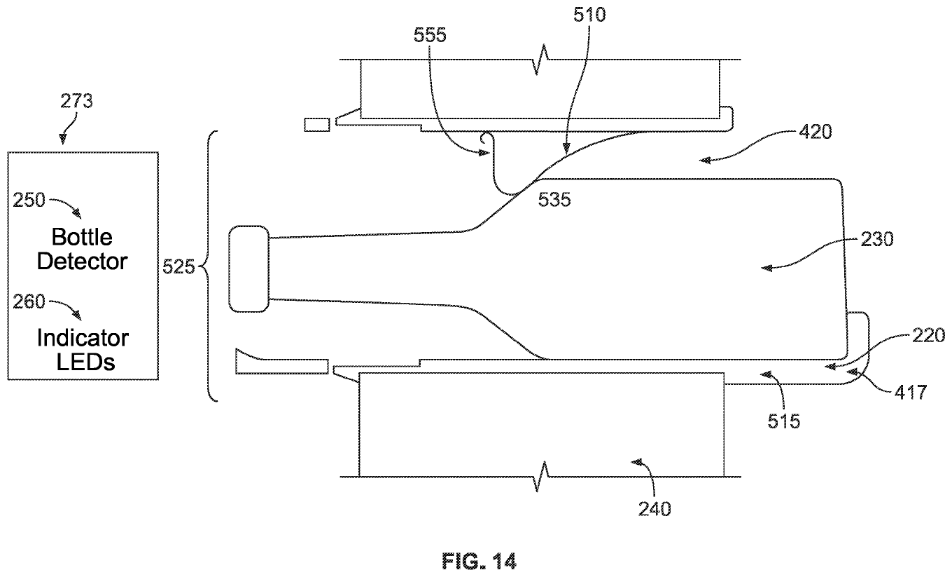

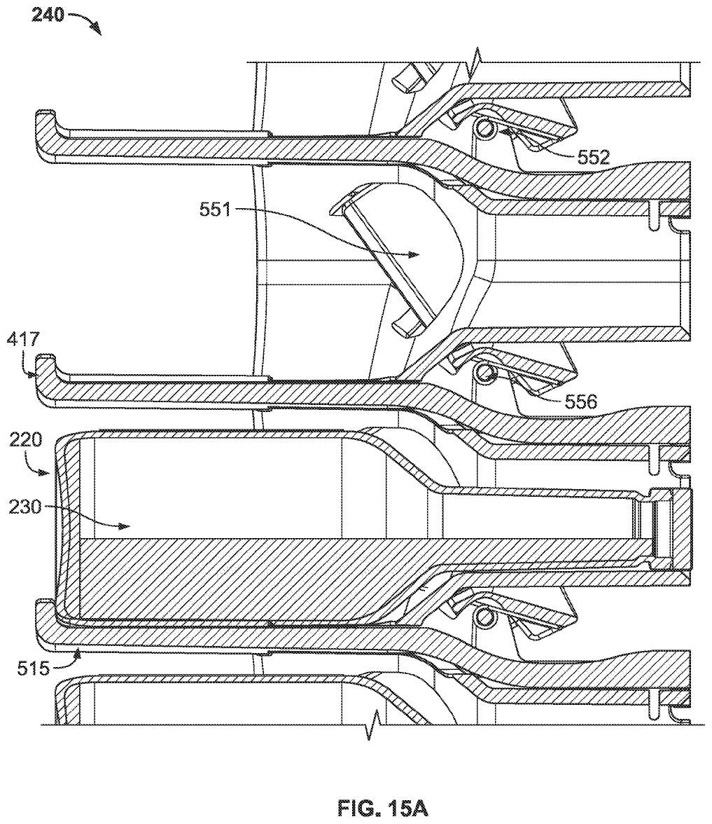

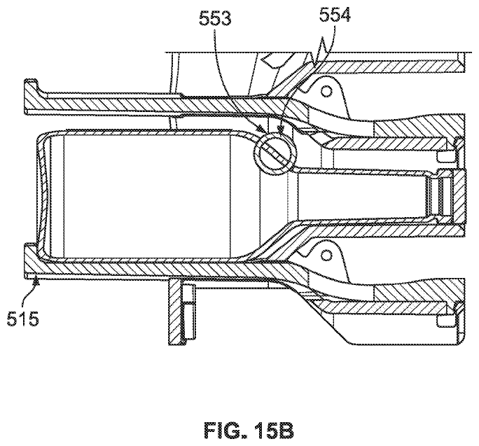

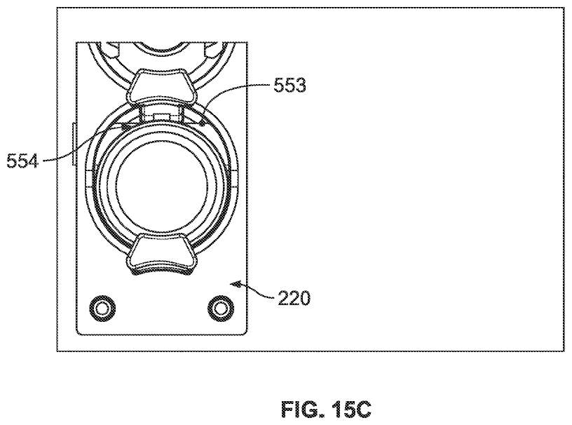

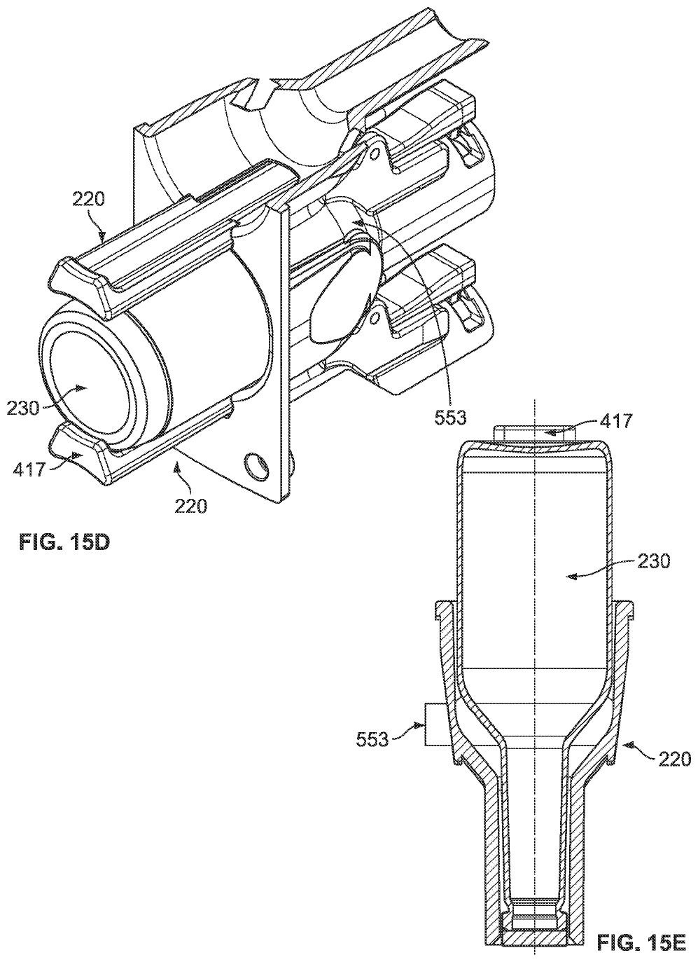

With reference to FIG. 14, the receptacle is illustrated with the light pipe 515 to translate the light from indicator LEDs 260 from the distal end 525 of the receptacle (i.e., the inside of the drum 240 in which the receptacle is disposed). The light pipe 515, if present, cradles the bottle 230 and extends past the proximal end 420 of the receptacle (i.e., the outer surface of the drum 240). The flat spring 510 presses against the upper shoulder 535 of the culture bottle 230 to hold the culture bottle 230 against the tab 417. The receptacle 220 is adjacent to a bottle presence detector 250 at the distal end 525 of the receptacle 220. The distal end of a bottle 230 in receptacle 220 is detected by bottle presence detector 250. The bottle presence detector 250 is carried by the status indication board 273. As illustrated in FIG. 15A-15E, the spring is enmolded into the bottle holder 220.

The light pipe 515 lines up with indicator LEDs 260 on the status indication board 273. The surface of the end of the light pipe 515 outside the bottle drum 240 is textured to disperse the light from the indicator LEDs 260. The bottle crimp cap 410 interrupts the bottle presence detector 250 (e.g., an optical switch or a proximity sensor) when it is placed in the receptacle 220. The indicator LEDs 260 and bottle presence detector 536 are located on the status indication board 273 that is positioned on the inside of the drum 240 in an arrangement that corresponds to each bottle 230 in the drum 240 that is accessible by the user. The bottle presence detectors 250 are monitored while a door to the module is open to detect in real time when a bottle 230 is placed in a receptacle or removed from a receptacle.

FIG. 15A illustrates, a portion of a drum 240, with multiple vertical rows of the receptacles 220. The drum is illustrated in cutaway view to show the culture bottle 230 support in receptacles 220. The top receptacle 220 is empty. In the embodiment illustrated in FIG. 15A, a pivoting arm 551 is provided to secure the culture bottle 230 in the receptacle 220 instead of the leaf spring 550 previously described. As the bottle 230 is advanced into the receptacle 230, the pivoting arm 551 rotates clockwise to secure the culture bottle 230 in the receptacle. Resistance to pivoting arm 551 is applied by coil spring 552, which is secured in the receptacle with a pin 556.

An alternative to the receptacle illustrated in FIG. 15A is illustrated in FIGS. 15B to FIG. 15E. Referring to FIG. 15B, the pivoting arm 551 illustrated in FIG. 15A is replaced by a deformable material 553. In one example, the deformable material 553 is a peristaltic tubing but other conventional deformable materials are contemplated. A key aspect of the deformable material is its resilience in assuming its undeformed shape after each instance of being deformed by insertion of the culture bottles in the receptacle. The bottom portion of the receptacle 220 is light pipe 515.

The deformable material 553 is placed in tapered portion 554 of the receptacle 220. Referring to FIG. 15C, an end view of the receptacle (220) illustrates the deformable material 553 at the top portion of the receptacle, (along the tapered portion 554 of the receptacle). Suitable deformable materials include, in addition to the elastomeric peristaltic tubing described above, elastomeric materials and foam materials.

FIG. 15D is perspective view of one receptacle 220 with a portion of second receptacle formed above it. The culture bottle is retained in the receptacle as described above. Tab 417 retains the culture bottle 230 in the receptacle 220. Other materials for the deformable material are contemplated as having sufficient frictional properties when used in contact with bottle 230. Such friction prevents the rotation of bottle 230, thus allowing the measurement system to obtain high quality signals with less noise caused by bottle vibration due to rack movement. FIG. 15E is a top down view of the culture bottle retained in the receptacle 220 with the light pipe 515.

In one aspect, the module cabinets in which the drum racks are placed may have a heat distribution system made of a lightweight moldable expandable polypropylene (EPP) foam. This is referred to as a molded foam heat distribution assembly herein. While EPP foam is described herein, other moldable synthetic materials, such as expandable polystyrene (EPS), are contemplated. EPP foam is more elastic and less brittle than EPS, making it better suited in applications where the foam may be subjected to operational stresses. One skilled in the art may select a suitable moldable foam for the molded foam heat distribution assembly described herein.

A molded foam heat distribution assembly is customizable, which is an advantage when one wishes to provide an ambient heated air environment that is relatively uniform (i.e., relatively devoid of temperature gradients). The customized heating environment may be tailored to ensure that bottles in the rack do not have different temperatures and that the ambient temperature and the actual temperature of individual bottles in the rack are essentially uniform (with accepted tolerances, which are ±about five percent). That is, the ambient temperature in the cabinet should not vary more than ±about five percent at any location in the cabinet and the temperatures of individual bottles in the rack (at equilibrium within the heated cabinet) should not vary more than about ±about five percent. For example, if the target ambient temperature of the incubator is 36.5° C., then the ambient temperature may vary by ±1° C., which is a ±2.7% tolerance when incubating one or more drum racks that are completely full (e.g., 240 bottles). The ambient temperature and the bottle temperature will have a wider degree of variation with each other because it takes time for the ambient heat to raise the bottle temperature to the ambient heat temperature.

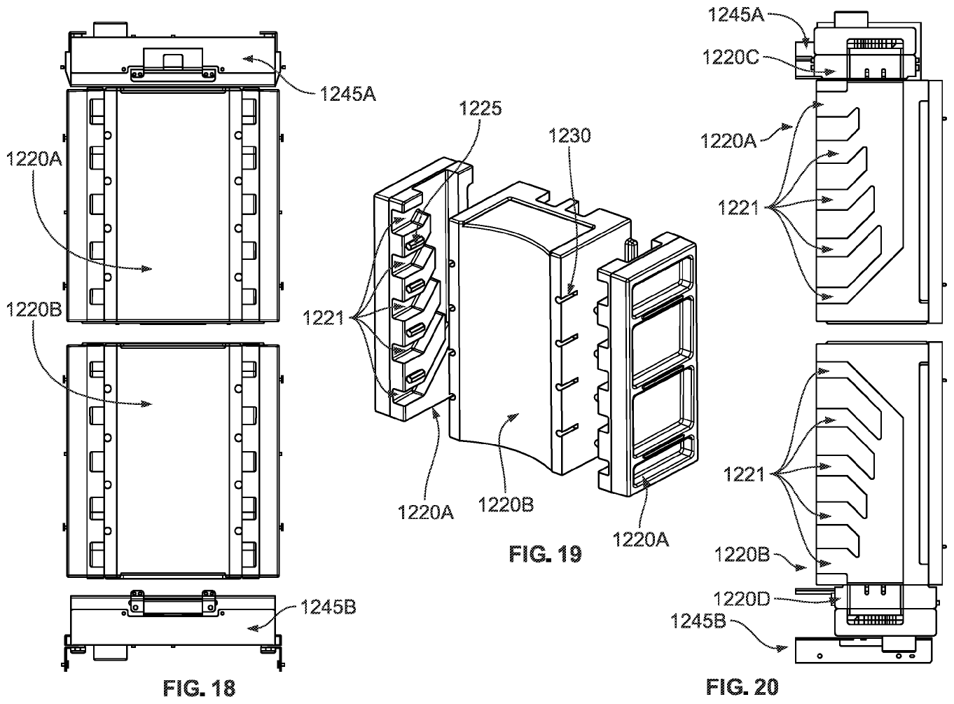

In a further aspect, the one or more heater/blower units (one unit for each cabinet) have a motor-driven blower that blow a sufficient volume of air over a heater (e.g., a 250-watt heater) and dispenses the hot air through an outlet opening and directed onto the rotating drum rack (with the culture bottles therein). The hot air is directed onto different rows of the rotating drum rack by a plurality of ducts formed in the molded EPP foam inserts. The heater/blower units are assembled in a two-cabinet system such that one unit is below the rotating drum rack in the bottom cabinet and the other unit is place above the rotating drum rack in the top cabinet. This arrangement is illustrated in FIG. 18, which is described in detail hereinbelow. In one aspect, the heater/blower units are installed on rails to make them easy to service. The units are held in place by leaf springs to hold them in position. In one aspect, as illustrated in FIGS. 18-20 below, there are five (5) ducts 1221 molded into the side inserts 1220A. The ducts 1221 provide more targeted airflow so that all bottles in the rack are approximately uniformly exposed to the heated, blown air. Blowing hot air through this air distribution system provides for a relatively uniform temperature distribution throughout the cabinet is which the rotating drum rack is placed. Specifically, the ducts provide more uniform air distribution, and the blower provides circulating air that also distributes the warmed air throughout the cabinet, ensuring more uniform heating of the bottles carried in the rotating drum rack.

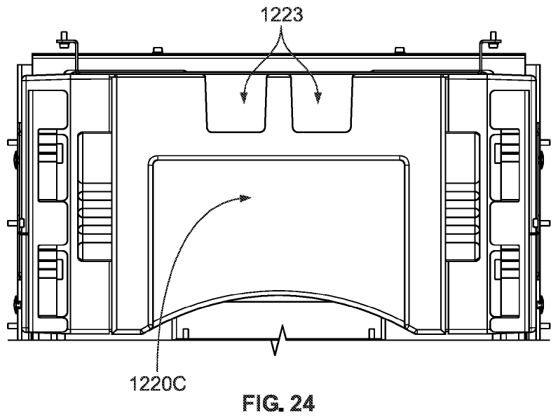

In one optional aspect, the molded foam insert heat distribution assembly may have additional ducts, such as ducts in the rear of the molded foam insert heat distribution assembly. Rear ducts 1223 are illustrated in FIG. 24, which is a top-down view of the molded foam insert heat distribution system above the top-component 1220C.

In one aspect, the motor for the heater/blower unit is a DC input centrifugal blower. Such a blower has a suitably compact design and sufficiently small exhaust that cooperates with the molded foam insert heat distribution assembly to provide suitable air flow into the assembly. The blower itself is placed in the molded foam insert heat distribution assembly in one aspect. The heater and RTDs may also be received into the molded insert heat distribution assembly in a compartment with an outlet opening that is in fluid communication with the air duct inlets of the molded insert heat distribution assembly. To mitigate the loss of heat and air, the molded foam insert heat distribution assembly is secured together in a conventional manner. In one aspect, molded foam panels from which the assembly is formed are secured to each other and to the module frame using snap fits and sheet metal clam shells. The clam shells are secured together using screws, for example. In addition to providing thermal insulation to the interior of the module, the molded foam insert heat distribution assembly mitigates sound emanating from the module and dampens vibrations from both the interior and exterior of the module.

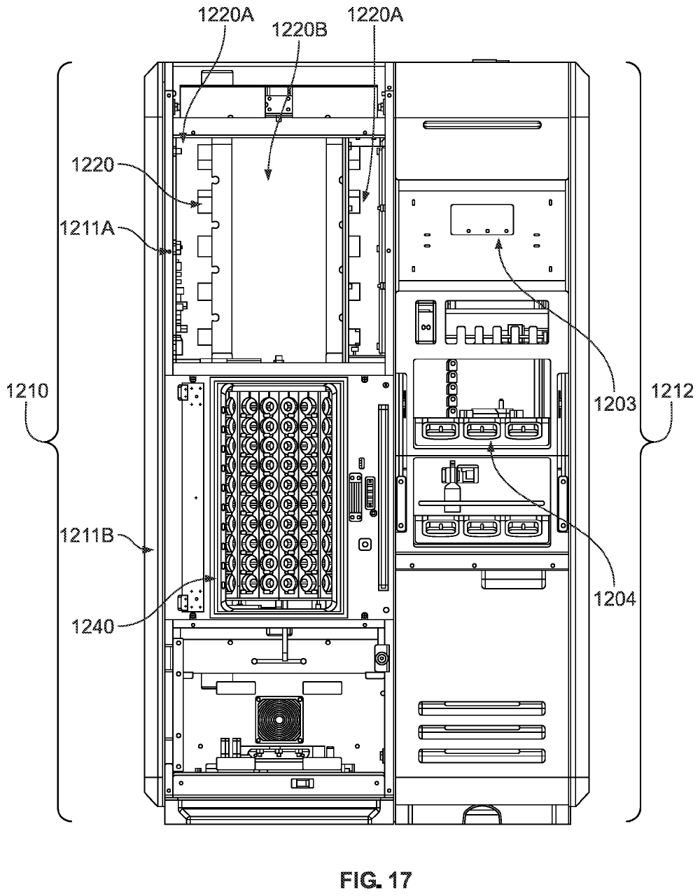

Regarding the addition of the molded foam insert heat distribution assembly to provide ducts for circulating heated air past the drum, the assembly is formed at the back of the frame that receives the drum rack. FIG. 17 illustrates a module 1210 according to one aspect of the apparatus for incubating, storing and monitoring the blood culture bottles described herein. The module 1210 has two cabinets 1211, each for receiving a drum rack 1240 as described elsewhere herein. In FIG. 17, as illustrated, cabinet 1211A does not have a drum rack placed therein (but can receive one) while cabinet 1211B does have a drum rack 1240 therein. When the module 1210 is in operation, a drum rack 1240 is in each cabinet. The module 1210 is illustrated alongside a different module 1212. Module 1212 has a touch screen 1203 and an input/output rack 1204 for receiving culture bottles into the module 1212 from where they are conveyed into module 1210. Module 1212 is described in U.S. Provisional Application 63/390,535, which was filed on Jul. 19, 2022, and is incorporated by reference herein.

Compartment 1211A has a foam insert heat distribution assembly 1220 therein. The foam insert heat distribution assembly 1220 is formed from molded side duct portions 1220A and a concave middle section 1220B. The concave middle section 1220B is formed to accommodate the curvature of the drum rack 1240, and to allow the air ducts 1221 to be as close to the drum rack 1240 as possible without interfering with drum rotation.

FIG. 18 is a view of the molded foam insert heat distribution assembly in communication with heater/blower assemblies 1245A/1245B for the module 1210 without other module components. For a two-cabinet module, there is the molded foam insert heat distribution assembly 1220A for cabinet 1211A and the molded foam insert heat distribution assembly 1220B for the cabinet 1211B. Each molded foam insert heat distribution assembly is in fluid communication with a heater/blower assembly 1245A and 1245B, respectively. Referring to FIGS. 18-20, the two-cabinet construction has heater/blower 1245A and molded foam insert heat distribution assembly 1220A as the top cabinet and heater/blower 1245B and molded foam insert heat distribution assembly 1220B as the lower cabinet. Their orientations are different, with heater/blower assembly 1245B in an orientation that is upside down when compared with the orientation of heater/blower assembly 1245A. With reference to FIG. 20, it is observed that the molded foam insert heat distribution assembly 1220B is a mirror image of the molded foam insert heat distribution system 1220A. Although received into the module in different orientations, the components are interchangeable. This provides ease and efficiency of manufacturing since the components are uniform.

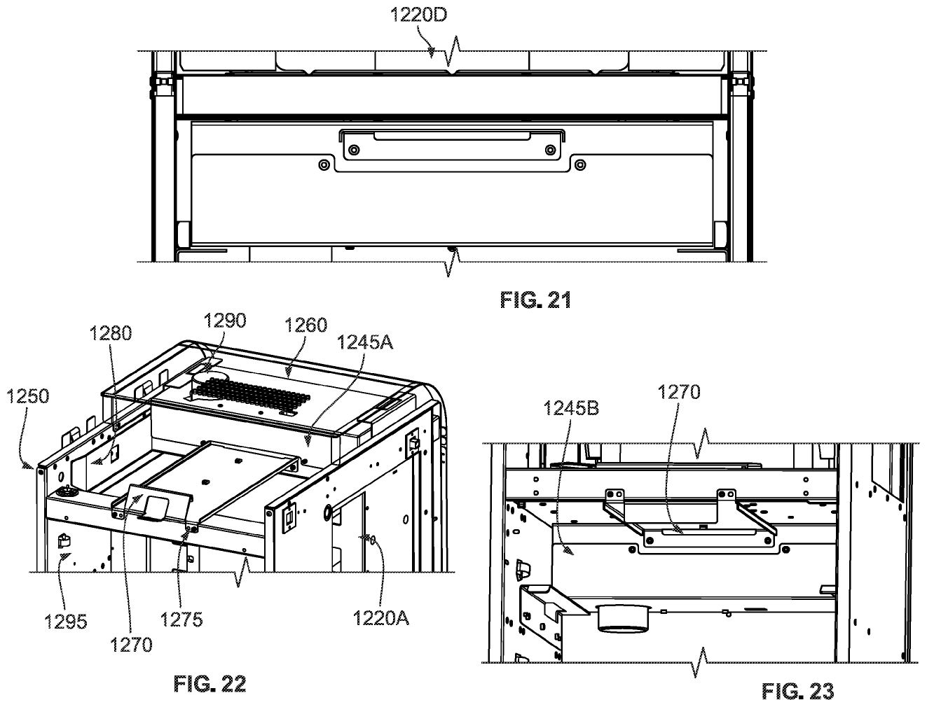

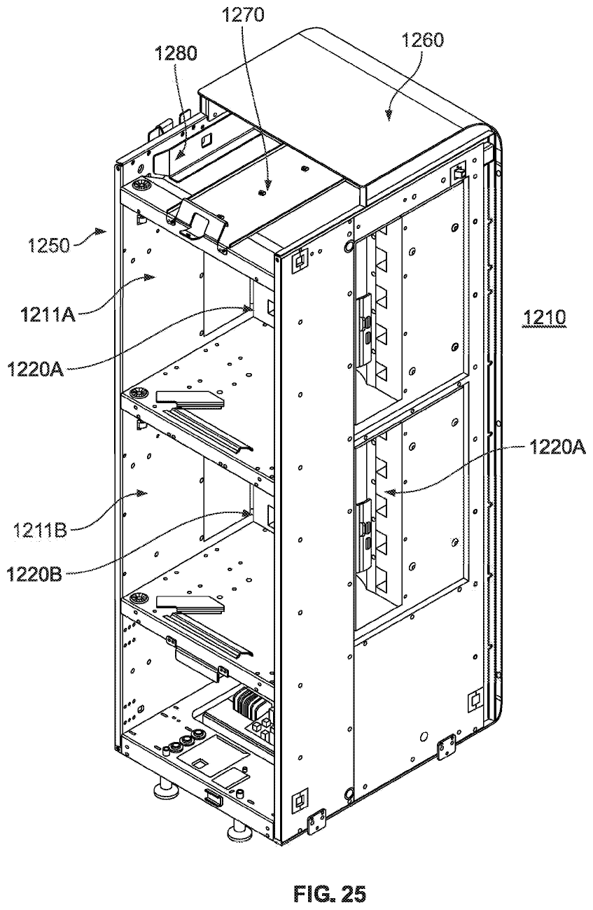

Referring to FIG. 25, the frame 1250 of module 1210 is illustrated without the rotating drum racks placed therein. The molded foam insert heat distribution assemblies 1220A and 1220B are placed in the rear of the compartments 1211A and 1211B. Also illustrated in FIG. 25 and FIG. 22 is a housing 1260 into which is received the heater/blower assembly 1245A (only the top heater/blower assembly is visible in FIGS. 22 and 25). The bottom heater/blower assembly 1245B is illustrated in FIGS. 21 and 23. To allow for access to the heater/blower assembly 1245, there is provided a long handle 1270 that extends to the front of the frame 1250. Rails 1280 are provided so that the heater/blower assembly 1245A/1245B can be easily slid out of the housing 1260 for service using handle 1270. With reference to FIG. 22, the housing 1260 is shown in phantom so that the heater/blower assembly 1245A is visible therethrough. The heater/blower assembly 1245A is held in the housing 1260 by spring clips 1290. When the handle 1270 is moved forward, the heater/blower assembly 1245A is advanced out of the housing 1260 for service. The handle 1270 is secured onto the frame by screws 1275. FIG. 23 illustrates the heater/blower assembly 1245B and the handle 1270 that is used to pull heater/blower assembley 1245B from its housing.

The molded foam insert heat distribution assemblies 1220A and 1220B provide several advantages. Specifically, they can be assembled and placed in the module with a minimum of additional parts such as sheet metal and fasteners, which are used in more conventional heat distribution systems. In one aspect, a molded foam insert heat distribution system is an assembly of five molded foam pieces. Those are the three components 1220A, 1220B, and 1220A illustrated in FIG. 19, which form the integrated air ducts, along with a top component 1220C (FIG. 24) and a bottom component 1220D (FIG. 21). As illustrated in FIG. 19, component 1220A has tabs 1225 which are received by notches 1230 in component 1220B. This allows for assembly of the components 1220A to the component 1220B by inserting the tabs 1225 into the notches 1230. The air distribution system formed from the molded foam provides for an air flow with less turbulence. Because the molded foam inserts fit together, putting the assembly together is easier and assembly errors are less likely to occur. In a further aspect, the ducts 1221 are tapered to improve airflow. The tapered ducts 1221 are illustrated in FIGS. 19 and 20. In addition to the tapered ducts 1221, the molded foam heat distribution assembly has smaller ducts/vents to provide more heated air flow paths that can be directed toward more bottles carried by the rack 1240.

In one aspect, the module 1210 has a molded foam insert heat distribution assembly that may be assembled simply by joining the pieces together. In one aspect, the individual molded components may have tongue in groove features to facilitate assembly. In one aspect, five pieces are assembled to form one molded foam insert heat distribution assembly. Ease of installation can be further enhanced using crush ribs (allowing larger objects to be accepted into a smaller opening), Christmas tree fasteners, etc. One skilled in the art is familiar with suitable fasteners that may be used to assemble the molded foam insert heat distribution assembly together and such fasteners are not described in detail herein. The molded foam insert heat distribution assembly is fluidically coupled to a heater/blower unit 1245A/1245B as described herein.

The heater/blower unit 1245A/1245B, in one aspect, has a lightweight design. Features such the handle, side rails and leaf spring allow the heater/blower unit to be easily insert into, held securely by, and easily removed from the module. The leaf springs also secure the heater/blower unit so that hot air leakage between the blower and the molded foam insert heat distribution assembly is mitigated. The handle 1270 can also be used to anchor/secure cables 1295 to the heater/blower 1245.

The molded foam insert heat distribution assembly, as noted above, allows for targeted air flow distribution.

As explained herein, the module rotates the drum both for positioning the bottles for user access and also for automation access. The module also rotates the culture bottles to agitate them.

The apparatus described herein provides the following advantages: 1) a reduction of noise (i.e., the ratio of growth signal to reference signal should be unaffected by bottle position, temperature, and sensor variability); 2) detection of growth in a vial that experiences a delay in entry into the system (i.e., the dual measurements described above provide a reference such that the contents of the vial do not need to be sampled continuously during growth to confirm positivity by detecting growth acceleration); and 3) signal quality indicator (i.e., the reference signal is an independent indicator of the health of the station hardware).

Described herein is an apparatus for storing and monitoring blood culture bottles. The apparatus has a frame defining at least one cabinet. The cabinet may receive a drum-shaped rack having an exterior perimeter and an interior perimeter therein. The exterior perimeter has a diameter in excess of a diameter of the interior perimeter. The drum has a plurality of receptacles, the receptacles having a proximal end at the exterior perimeter and a distal end at the interior perimeter. Each receptacle may receive a blood culture bottle, which has a bottom portion and a neck portion. The bottle can be received by the receptacle such that either the bottom portion is received at the distal end of the receptacle or the neck portion is received at the distal end of the receptacle. In one aspect, the drum perimeters are disposed about an axis of rotation of the drum.

The plurality of receptacles may be arranged in the drum as an array of receptacles, the array having receptacles disposed both vertically and horizontally. The vertically aligned receptacles may form a column. The horizontally aligned receptacles may form a row.

Each cabinet may have a molded foam insert heat distribution assembly therein. In one aspect, the molded foam insert assembly may be molded foam inserts assembled together. Each molded foam insert heat distribution assembly may be in fluid communication with a heater/blower assembly. In one aspect, the frame is disposed in a housing.

In one aspect, the cabinet in the apparatus may have two molded foam side inserts that are assembled to a molded foam central insert. In a further aspect, the molded foam side insert, when assembled to the molded foam central insert, may define a plurality of ducts.

In a further aspect, the heater/blower assembly is in fluid communication with the ducts. In a further aspect, a duct outlet is directed toward a receptacle in the drum-shaped rack. In a further aspect, the heater/blower assembly is supported by the frame either above the cabinet in which the molded foam insert heat distribution is placed or below the cabinet in which the molded foam insert heat distribution is placed. In a further aspect, the heater/blower assembly is placed in a housing.

In a further aspect, the frame has a handle and rails to advance the heater/blower assembly into the housing and remove it therefrom.

In another aspect, described herein is an apparatus for storing and monitoring blood culture bottles. The apparatus may have a drum-shaped rack having an exterior perimeter and an interior perimeter therein. The exterior perimeter may have a diameter in excess of a diameter of the interior perimeter. The drum may have a plurality of receptacles, the receptacles having a proximal end at the exterior perimeter and a distal end at the interior perimeter. Each receptacle may be configured to receive a blood culture bottle. In one aspect, the blood culture bottle has a bottom portion and a neck portion. The bottle may be received by the receptacle such that either the bottom portion is received at the distal end of the receptacle or the neck portion is received at the distal end of the receptacle. In a further aspect, the drum perimeters are disposed about an axis of rotation of the drum and a drive motor may be placed within the interior perimeter of the drum in line with the axis of rotation. In a further aspect, the apparatus may have a drum motor encoder rigidly coupled to a shaft of a motor shaft of the drive motor.

The apparatus may have alignment sensors adapted to detect when the axis of rotation of the drum is not a vertical axis. In a further aspect, the apparatus may have bottle presence sensors that detect the presence of a bottle in a receptacle. The bottle presence sensors may be adapted to detect an aligned state of the drum and a nonaligned state of the drum. The unaligned state may be detected when a bottle presence sensor fails to detect a bottle known to be presently in a receptacle opposite the non-detecting sensor. In a further aspect, the drum motor encoder may provide feedback to an operator when the operator is moving the drum rack manually. The drum motor encoder may increment the manual movement of the drum rack, so that the drum rack advances a predetermined degree of rotation upon manual rotation.

Described herein is an apparatus for storing and monitoring blood culture bottles, the apparatus having a frame defining at least one cabinet, the cabinet adapted to receive a drum-shaped rack having an exterior perimeter and an interior perimeter therein, the exterior perimeter having a diameter in excess of a diameter of the interior perimeter, the drum having a plurality of receptacles, the receptacles having a proximal end at the exterior perimeter and a distal end at the interior perimeter, each receptacle configured to receive a blood culture bottle, the blood culture bottle comprising a bottom portion and a neck portion, wherein the bottle can be received by the receptacle such that either the bottom portion is received at the distal end of the receptacle or the neck portion is received at the distal end of the receptacle. The drum perimeters are disposed about an axis of rotation of the drum. The plurality of receptacles are arranged in the drum as an array of receptacles, the array having receptacles disposed both vertically and horizontally, vertically aligned receptacles forming a column and the horizontally aligned receptacles forming a row. The apparatus also has a molded foam insert heat distribution assembly in each cabinet, the molded foam insert assembly comprising molded foam inserts assembled together. Each molded foam insert heat distribution assembly is in fluid communication with a heater/blower assembly. The frame is disposed in a housing.

The apparatus wherein the two molded foam side inserts are assembled to a molded foam central insert. In one aspect, the molded foam side insert, when assembled to the molded foam central insert, defines a plurality of ducts. In a further aspect, the heater/blower assembly is in fluid communication with the ducts. In one aspect, a duct outlet is directed toward a receptacle in the drum-shaped rack. In a further aspect, the heater/blower assembly is supported by the frame either above the cabinet in which the molded foam insert heat distribution is placed or below the cabinet in which the molded foam insert heat distribution is placed. In a further aspect, the heater/blower assembly is placed in a housing. In a further aspect, the frame comprises a handle and rails to advance the heater/blower assembly into the housing and remove it therefrom.

Described herein is an apparatus for storing and monitoring blood culture bottles. The apparatus has a drum-shaped rack having an exterior perimeter and an interior perimeter therein, the exterior perimeter having a diameter in excess of a diameter of the interior perimeter, the drum having a plurality of receptacles, the receptacles having a proximal end at the exterior perimeter and a distal end at the interior perimeter, each receptacle configured to receive a blood culture bottle, the blood culture bottle comprising a bottom portion and a neck portion, wherein the bottle can be received by the receptacle such that either the bottom portion is received at the distal end of the receptacle or the neck portion is received at the distal end of the receptacle. In the above-described apparatus, the drum perimeters are disposed about an axis of rotation of the drum. In a further aspect, a drive motor is placed within the interior perimeter of the drum in line with the axis of rotation. The apparatus also has a drum motor encoder rigidly coupled to a shaft of a motor shaft of the drive motor.

In the above apparatus, in one aspect, the apparatus has alignment sensors adapted to detect when the axis of rotation of the drum is not a vertical axis. The apparatus further comprises bottle presence sensors that detect the presence of a bottle in a receptacle. The bottle presence sensors are adapted to detect an aligned state of the drum and a nonaligned state of the drum. In one aspect, an unaligned state is detected when a bottle presence sensor fails to detect a bottle known to be presently in a receptacle opposite the non-detecting sensor. In the above apparatus, the drum motor encoder provides feedback to an operator when the operator is moving the drum rack manually. In a further aspect, the drum motor encoder increments the manual movement of the drum rack, so that the drum rack advances a predetermined degree of rotation upon manual rotation. In one aspect, the above drum-shaped rack is vertically movable relative to the drive motor. The drum-shaped rack may have a lifter for raising the drum relative to the motor. The drum-shaped rack described herein may have a locking tool for supporting the drum-shaped rack on a frame in which the drum-shaped rack is disposed when the drum is in a raised position. The apparatus described above may have a measurement board having sensors for determining status of the blood culture bottle as the drum-shaped rack is moved past the measurement board. As described throughout, the measurement board may be placed adjacent the exterior perimeter of the drum-shaped rack.

The above apparatus with the locking tool may have a locking tool with a handle and a bracket. In the above apparatus, the lifter may have a screw that advances through a motor assembly support plate and a guide nut. The above apparatus operates such that, when the drum-shaped rack is advanced upward, the screw forces a plate upward. In the above aspect, the plate travels upward along guide pins. In this aspect, the plate travels upward, the drum-shaped rack is raised off of drum supports, thereby creating a lift space between the frame and a rotor of the drive motor.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive.

It will further be understood that any reference herein to subject matter known in the field does not, unless the contrary indication appears, constitute an admission that such subject matter is commonly known by those skilled in the art to which the present technology relates.

1. An apparatus for storing and monitoring blood culture bottles, the apparatus comprising:
a frame defining at least one cabinet, the cabinet adapted to receive a drum-shaped rack having an exterior perimeter and an interior perimeter therein, the exterior perimeter having a diameter in excess of a diameter of the interior perimeter, the drum having a plurality of receptacles, the receptacles having a proximal end at the exterior perimeter and a distal end at the interior perimeter, each receptacle configured to receive a blood culture bottle, the blood culture bottle comprising a bottom portion and a neck portion, wherein the bottle can be received by the receptacle such that either the bottom portion is received at the distal end of the receptacle or the neck portion is received at the distal end of the receptacle;
wherein the drum perimeters are disposed about an axis of rotation of the drum;
wherein the plurality of receptacles are arranged in the drum as an array of receptacles, the array having receptacles disposed both vertically and horizontally, vertically aligned receptacles forming a column and the horizontally aligned receptacles forming a row;
a molded foam insert heat distribution assembly in each cabinet, the molded foam insert assembly comprising molded foam inserts assembled together;
wherein each molded foam insert heat distribution assembly is in fluid communication with a heater/blower assembly; and
wherein the frame is disposed in a housing.

2. The apparatus of claim 1, wherein two molded foam side inserts are assembled to a molded foam central insert.

3. The apparatus of claim 2, wherein the molded foam side insert, when assembled to the molded foam central insert, defines a plurality of ducts.

4. The apparatus of claim 1, wherein the heater/blower assembly is in fluid communication with the ducts.

5. The apparatus of claim 1, wherein a duct outlet is directed toward a receptacle in the drum-shaped rack.

6. The apparatus of claim 1, wherein the heater/blower assembly is supported by the frame either above the cabinet in which the molded foam insert heat distribution is placed or below the cabinet in which the molded foam insert heat distribution is placed.

7. The apparatus of claim 1, wherein the heater/blower assembly is placed in a housing.

8. The apparatus of claim 1, wherein the frame comprises a handle and rails to advance the heater/blower assembly into the housing and remove it therefrom.

9. An apparatus for storing and monitoring blood culture bottles, the apparatus comprising:
a drum-shaped rack having an exterior perimeter and an interior perimeter therein, the exterior perimeter having a diameter in excess of a diameter of the interior perimeter, the drum-shaped rack having a plurality of receptacles, the receptacles having a proximal end at the exterior perimeter and a distal end at the interior perimeter, each receptacle configured to receive a blood culture bottle, the blood culture bottle comprising a bottom portion and a neck portion, wherein the bottle can be received by the receptacle such that either the bottom portion is received at the distal end of the receptacle or the neck portion is received at the distal end of the receptacle;
wherein the drum perimeters are disposed about an axis of rotation of the drum;
wherein a drive motor is placed within the interior perimeter of the drum-shaped rack in line with the axis of rotation; and
a drum motor encoder rigidly coupled to a shaft of a motor shaft of the drive motor.

10. The apparatus of claim 9, further comprising alignment sensors adapted to detect when the axis of rotation of the drum is not a vertical axis.

11. The apparatus of claim 9, wherein the apparatus further comprises bottle presence sensors that detect the presence of a bottle in a receptacle, wherein the bottle presence sensors are adapted to detect an aligned state of the drum and a nonaligned state of the drum.

12. (canceled)

13. The apparatus of claim 11, wherein an unaligned state is detected when a bottle presence sensor fails to detect a bottle known to be presently in a receptacle opposite a non-detecting sensor.

14. The apparatus of claim 9, wherein the drum motor encoder provides feedback to an operator when the operator is moving the drum rack manually and increments manual movement of the drum rack, so that the drum rack advances a predetermined degree of rotation upon manual rotation.

15. (canceled)

16. The apparatus of claim 9, wherein the drum-shaped rack is vertically movable relative to the drive motor and wherein the drum-shaped rack comprises a lifter for raising the drum relative to the motor.

17. (canceled)

18. The apparatus of claim 9, wherein the apparatus comprises a locking tool for supporting the drum-shaped rack on a frame in which the drum-shaped rack is disposed when the drum is in a raised position.

19. The apparatus of claim 9, wherein the apparatus further comprises a measurement board comprising sensors for determining status of the blood culture bottle as the drum-shaped rack is moved past the measurement board, wherein the measurement board may be placed adjacent the exterior perimeter of the drum-shaped rack.

20. (canceled)

21. The apparatus of claim 18, wherein the locking tool comprises a handle and a bracket.

22. The apparatus of claim 16, wherein the lifter comprises a screw that advances through a motor assembly support plate and a guide nut.

23. The apparatus of claim 17, wherein, when advanced upward, the screw forces a plate upward, wherein the plate travels upward along guide pins

24. (canceled)

25. The apparatus of claim 23, wherein, when the plate travels upward, the drum-shaped rack is raised off of drum supports, thereby creating a lift space between the frame and a rotor of the drive motor.
